(19) United States
(12) Patent Application Publication
(10) Pub. No.: US 2025/0359524 A1
(43) Pub. Date: Nov. 27, 2025

(54) Systems For Use In Pollinating Plants, And Related Methods

(71) Applicant: Monsanto Technology LLC, Saint Louis, MO (US)

(72) Inventors: Eric Lee BORROWMAN, Saint Peters, MO (US); Justin Scott KAYSER, Wentzville, MO (US); Chad Allen STENDAL, O'Fallon, MO (US)

(21) Appl. No.: 19/215,143

(22) Filed: May 21, 2025

Related U.S. Application Data
(60) Provisional application No. 63/651,913, filed on May 24, 2024.

(51) Int. Cl. A01H 1/02

(52) U.S. Cl. CPC A01H 1/027

(57) ABSTRACT

Pollination assemblies for use in transferring pollen between plants are described herein. In one example embodiment, a pollination assembly includes a collection unit configured to dislodge pollen from pollen-bearing plants, where the collection unit includes a first air system configured to direct the dislodged pollen from the pollen-bearing plants to an outlet of the collection unit. The pollination assembly also includes at least one applicator unit configured to direct the dislodged pollen received from the pollen-bearing plants at pollen-receiving plants, and a distribution unit disposed adjacent the collection unit. The distribution unit includes a second air system configured to direct the dislodged pollen from the outlet of the collection unit to the at least one applicator unit.

### CROSS-REFERENCE TO RELATED APPLICATION

This application claims the benefit of, and priority to, U.S. Provisional Application No. 63/651,913, filed May 24, 2024. The entire disclosure of the above application is incorporated herein by reference.

### FIELD

The present disclosure generally relates to systems and methods for use in pollinating plants (e.g., collecting pollen, treating collected pollen, distributing pollen to desired plants, combinations thereof, etc.).

### BACKGROUND

This section provides background information related to the present disclosure which is not necessarily prior art.

In pollination, pollen grains are typically transferred from male anthers of plants (e.g., of flowers of the plants, etc.) to female stigmas (e.g., of flowers of the plants or other plants, etc.). The plants may be capable of self-pollination, cross-pollination, or both. Self-pollination involves the transfer of pollen from male anthers of plants (e.g., of flowers of the plants, etc.) to female stigmas of the same plants (e.g., of flowers of the same plants, etc.). And, cross-pollination involves the transfer of pollen from male anthers of plants (e.g., of flowers of the plants, etc.) to female stigmas of different plants (e.g., of flowers of different plants, etc.) (e.g., plants from a different family, line, etc.). In this way, the plants are able to create offspring in the form of seeds, which contain genetic information to produce new plants.

### SUMMARY

This section provides a general summary of the disclosure, and is not a comprehensive disclosure of its full scope or all of its features.

Example embodiments of the present disclosure generally relate to pollination assemblies for use in collecting and transferring pollen between plants. In one example embodiment, a pollination assembly generally includes a collection unit configured to dislodge pollen from pollen-bearing plants, the collection unit including a first air system configured to direct the dislodged pollen from the pollen-bearing plants to an outlet of the collection unit; at least one applicator unit configured to direct the dislodged pollen received from the pollen-bearing plants at pollen-receiving plants; and a distribution unit disposed adjacent the collection unit, the distribution unit including a second air system configured to direct the dislodged pollen from the outlet of the collection unit to the at least one applicator unit.

Example embodiments of the present disclosure also generally relate to pollination systems including the above pollination assembly. In one example embodiment, a pollination system includes a tractor having a carriage; and the pollination assembly coupled to the carriage of the tractor. In another example embodiment, a pollination system includes a rail system having at least one rail and a carriage configured for movement along the at least one rail; and the pollination assembly coupled to the carriage and moveable, via the carriage, along the at least one rail.

In another example embodiment, such a pollination assembly generally includes a collection unit configured to dislodge pollen from pollen-bearing plants, the collection unit including a first air system configured to direct the dislodged pollen from the pollen-bearing plants to an outlet of the collection unit; a storage unit; and a distribution unit disposed adjacent the collection unit, the distribution unit including a second air system configured to direct the dislodged pollen from the outlet of the collection unit to the storage unit.

Example embodiments of the present disclosure also generally relate to methods for collecting and transferring pollen between plants. In one example embodiment, such a method generally includes receiving at least one pollen-bearing plant into a collection unit of a pollination assembly; dislodging, by at least one agitator, pollen from the at least one pollen-bearing plant within the collection unit; directing, by a first air system, the dislodged pollen from the pollen-bearing plant to an outlet of the collection unit; receiving, by at least one distributor, the dislodged pollen from the outlet of the collection unit; decelerating the dislodged pollen within the at least one distributor; and directing, by at least a second air system, the dislodged pollen from the at least one distributor to at least one applicator unit for transfer to at least one pollen-receiving plant.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of a pollination system according to an example embodiment of the present disclosure, having a pollination assembly coupled to a tractor;

FIG. 2 is another perspective view of the pollination system of FIG. 1, with part of the tractor removed and with the pollination system schematically shown in a field;

FIG. 3 is another perspective view of the pollination system of FIG. 1, with part of the tractor removed;

FIG. 4 is a top plan view of the pollination system of FIG. 3;

FIG. 5 is a side elevation view of a collection unit and a distribution unit of the pollination assembly of FIG. 1;

FIG. 6 is a bottom plant view of the collection unit of FIG. 5;

FIG. 7 is a fragmentary front elevation view of the collection unit of FIG. 5;

FIG. 8 is a fragmentary side elevation view of an air conveyor of the pollination assembly of FIG. 1;

FIG. 9 is a top plan view of a manifold of the distribution unit of FIG. 5;

FIG. 10 is a perspective view of an applicator unit of the pollination assembly of FIG. 1;

FIG. 11 is a perspective of an example embodiment of an alternate applicator unit suitable for use with the pollination assembly of FIG. 1;

FIG. 12 is a schematic view of an example embodiment of a duct suitable for use with each of the applicator units of the pollination assembly of FIG. 1;

FIG. 13 is a perspective view of the pollination system of FIG. 1 with a carriage of the pollination system shown folded to allow for storage and/or transport of the pollination assembly;

FIG. 14 is perspective view of an example embodiment of a collection unit and a distribution unit that may be used with the pollination assembly of FIG. 1;

FIG. 15 is a fragmentary perspective view of the collection unit of FIG. 14;

FIG. 16 is another fragmentary perspective view of the collection unit of FIG. 14;

FIG. 17 is a fragmentary front elevation view of the collection unit of FIG. 14;

FIG. 18 is another fragmentary front elevation view of the collection unit of FIG. 14, illustrating movement of pollen into and through the collection unit;

FIGS. 19-20 are fragmentary perspective views of an example embodiment of an agitator that may be used in the collection unit of FIG. 14;

FIG. 21 is a top plan view of a pollination system according to another example embodiment of the present disclosure, having a pollination assembly coupled to a tractor;

FIG. 22 is a perspective view of an example embodiment of a pollen storage unit suitable for use with the pollination system of FIG. 21;

FIG. 23 is a fragmentary perspective view of an example embodiment of a pollination system including multiple pollination assemblies coupled to a tractor;

FIG. 24 is a side elevation view of the pollination system of FIG. 23;

FIG. 25 is a graph comparing mean percent of flat seeds produced using pollination assemblies of the present disclosure to pollinate pollen-receiving plants compared to mean percent of flat seeds produced not using pollination assemblies of the present disclosure to pollinate pollen-receiving plants;

FIG. 26 illustrates locations of different seeds on an example ear of corn;

FIG. 27 illustrates improved seed size distribution in an ear of corn as produced following pollination via the pollination assemblies of the present disclosure; and

FIG. 28 is a graph of seed purity showing a statistically significant increase to the purity of the seed that was created using the pollination assemblies and methods of the present disclosure.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

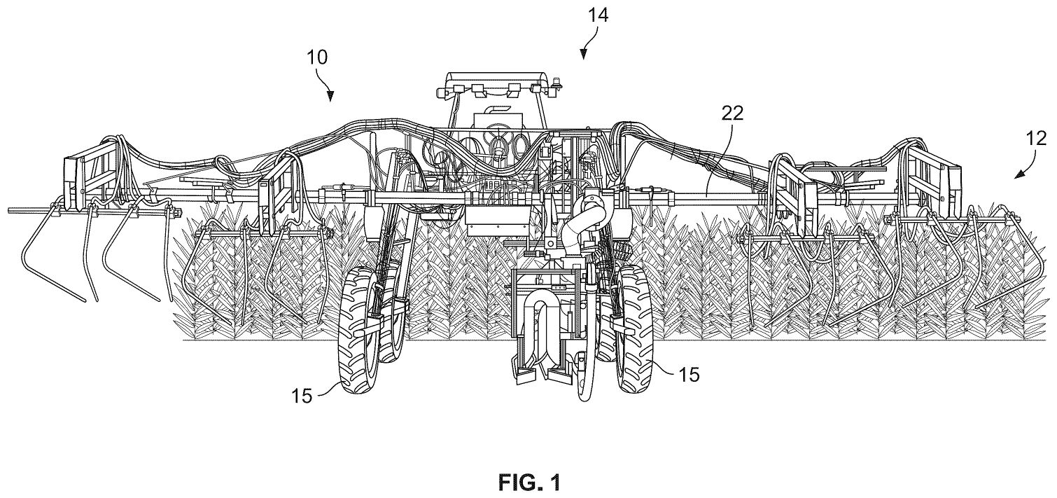

FIGS. 1-13 illustrate an example embodiment of a pollination assembly 10 including one or more aspects of the present disclosure. The pollination assembly 10 is configured to collect pollen from plants, and/or direct, move, deliver, convey, transfer, etc. pollen between plants, for example, for effecting pollination, etc. of the plants. In the illustrated embodiment, the pollination assembly 10 is shown in a field 12 (FIG. 1), for example, in combination with (e.g., coupled to, mounted on, etc.) a tractor 14, whereby the pollination assembly 10 operates to transfer the pollen between plants in the field 12 (in a targeted, precise manner, etc.) as the pollination assembly 10 moves through the field 12 (via the tractor 14, etc.). In this way, the pollination assembly 10 and the tractor 14 may be viewed as a pollination system. In other embodiments, the pollination assembly 10 may be used independent of the tractor 14, for example, where the pollination assembly 10 may be moveable on a rail system, or by other means, etc. to transfer pollen between plants in the field 12 (as the pollination assembly 10 moves through the field 12 along the rails, or other means, etc.). In addition, in still other embodiments, the pollination assembly 10 may be implemented/used in a greenhouse or other growing space to transfer pollen between plants therein (e.g., via a rail system, etc. in the greenhouse or other growing space, etc.).

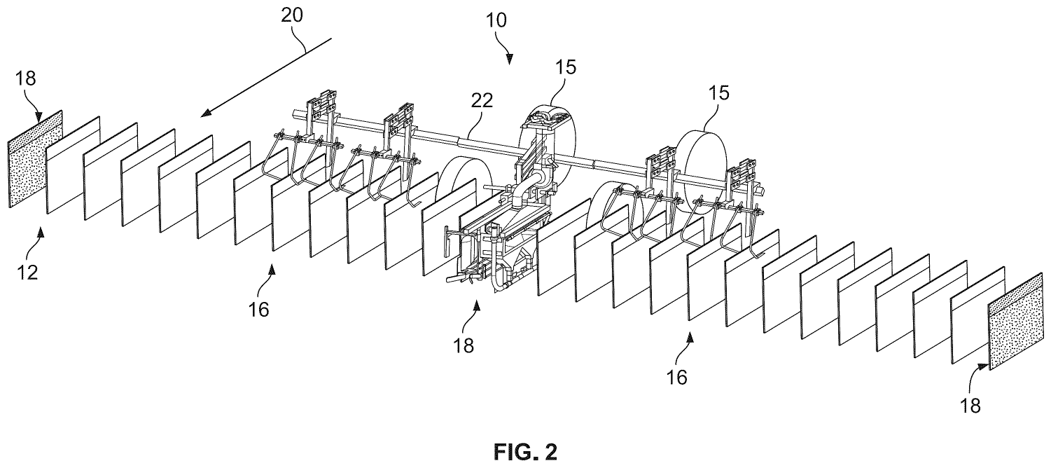

As shown in FIG. 2, the field 12 includes multiple rows of pollen-receiving plants 16 and multiple rows of pollen-bearing plants 18 (e.g., corn plants, etc.). More particularly, the illustrated field 12 includes spaced apart rows of pollen-bearing plants 18, and then groups of rows of pollen-receiving plants 16 located between each row of pollen-bearing plants 18. For instance, the illustrated field 12 includes a group of twelve rows of the pollen-receiving plants 16 spaced between each row of pollen-bearing plants 18 (e.g., a 12:1 (female/male) planting pattern, etc.). This arrangement may be repeated as desired in the field 12, for example, so that a group of the rows of pollen-receiving plants 16 is adjacent a row of pollen-bearing plants 18. In the example illustrated in FIG. 2, the field 12 includes a first row of pollen-bearing plants 18 (generally to the left of the field 12 in FIG. 2), a first group of rows of pollen-receiving plants 16, a second row of pollen-bearing plants 18 (generally in the middle of the field 12 in FIG. 2 at the pollination assembly 10), a second group of rows of pollen-receiving plants 16, and then a third row of pollen-bearing plants 18 (generally to the right of the field 12 in FIG. 2). In this arrangement, as illustrated, the generally middle row of pollen-bearing plants 18 may be used to pollinate at least some of pollen-receiving plants 16 in the first group and at least some of the pollen-receiving plants 16 in the second group. And, the generally left row of pollen-bearing plants 18 may then be used to pollinate the remaining pollen-receiving plants 16 in the first group, while the generally right row of pollen-bearing plants 18 may be used to pollinate the remaining pollen-receiving plants 16 in the second group.

In the illustrated embodiment, as described, the field 12 includes pollen-bearing plants 18 and pollen-receiving plants 16 arranged in a 12:1 (female/male) planting pattern. It should be appreciated, though, that the field 12 may be planted with different configurations of plants within the scope of the present disclosure. For instance, the field 12 may include groups of pollen-receiving plants 16 with more than or less than twelve rows of plants (e.g., 4:1 (female/male) planting patterns, 6:1 (female/male) planting patterns, 8:1 (female/male) planting patterns, etc.). In addition, or alternatively, the field 12 may include multiple rows of pollen-bearing plants 18 grouped together between rows of pollen-receiving plants 16, etc. (e.g., 12:2 (female/male) planting patterns, 8:2 (female/male) planting patterns, etc.). Further, the rows of plants may be spaced as desired within the field 12, for example, to accommodate (and receive) wheels 15 of the tractor 14 therebetween (e.g., the rows within the groups may be spaced apart by a distance of about 20 inches, by a distance of about 30 inches, by a distance of between about 15 inches and about 60 inches, by a distance of more than about 60 inches, by a distance of less than about 15 inches, etc.).

In this example embodiment, the plants 16, 18 include corn plants. In addition, the pollen-receiving plants 16 have been emasculated or otherwise modified (e.g., detasseled, etc.) such that the pollen-receiving plants 16 cannot pollinate themselves or other plants. Such emasculation may be done mechanically, chemically, or genetically, etc. After the pollen-receiving plants 16 are emasculated, they still can be pollinated by the pollen of the pollen-bearing plants 18. It should be appreciated that the pollination assembly 10 may be used with other plants, other than corn, within the scope of the present disclosure, for example, wheat, canola, tomato, eggplant, sweet and hot peppers, amaranth, barley, oat, rye, wild rice, walnut, pecan, cabbage, broccoli, spinach, other suitable plants, etc. In addition, in some example embodiments, the pollen-bearing plants 18 may include a first variety of plant and the pollen-receiving plants 16 may include a second variety of the same plant. As such, when the pollen-receiving plants 16 are pollinated with pollen from the pollen-bearing plants 18, the pollen-receiving plants 16 produce cross-pollinated seeds that may be used for growing a cross-pollinated variety of the crop plant with certain altered characteristics.

That said, in this example, the tractor 14 is configured to carry the pollination assembly 10 through the field 12 to pollinate the pollen-receiving plants 16 with the pollen collected/obtained from the pollen-bearing plants 18 (in one of the rows adjacent the pollen-receiving plants 16). In particular, the tractor 14 is positioned in the field 12 to drive along, or in the same direction as, the rows of the plants 16, 18, while supporting the pollination assembly 10 (e.g., with the wheels 15 of the tractor 14 positioned in the spacing between the rows of the pollen-receiving plants 16 and/or the rows of the pollen-bearing plants 18, etc.). The pollination assembly 10, then, is configured to collect, receive, obtain, etc. pollen from the pollen-bearing plants 18 (e.g., from tassels of the pollen-bearing plants 18, etc.) and direct (and deliver/transfer) the pollen to the adjacent pollen-receiving plants 16 (e.g., to female flowers of the pollen-receiving plants 16, etc.).

The tractor 14 is configured to travel along the rows of the plants 16, 18 in a generally forward direction (as generally indicated by arrow 20 in FIG. 2) (e.g., generally parallel to the rows of the plants 16, 18; etc.). The tractor 14 includes a carriage 22 (e.g., a tool bar, etc.) coupled to the tractor 14 adjacent a front portion of the tractor 14. The pollination assembly 10, then, is configured to couple to (e.g., mount to, etc.) the tractor 14 at the carriage 22, whereby the pollination assembly 10 is configured to move along the rows of the plants 16, 18 with the tractor 14. The tractor 14 may travel through the field 12 at desired speeds, for example, between about two miles per hour (mph) and about ten mph during operation (with the pollination assembly 10), etc. However, it should be appreciated that the tractor 14 may travel through the field 12 at other speeds within the scope of the present disclosure (e.g., at speeds greater than about ten mph, etc.). In the illustrated embodiment, the tractor 14 includes a high clearance farm tractor such as an applicator from Hagie Manufacturing Company of Clairon, IA. However, other tractors, or other vehicles or machines more generally, suitable for carrying the pollination assembly 10 through the field 12 may be used in other embodiments. For example, in some embodiments, the pollination assembly 10 may be coupled to a carriage 22 (broadly, a machine) arranged to move along a rail, etc.

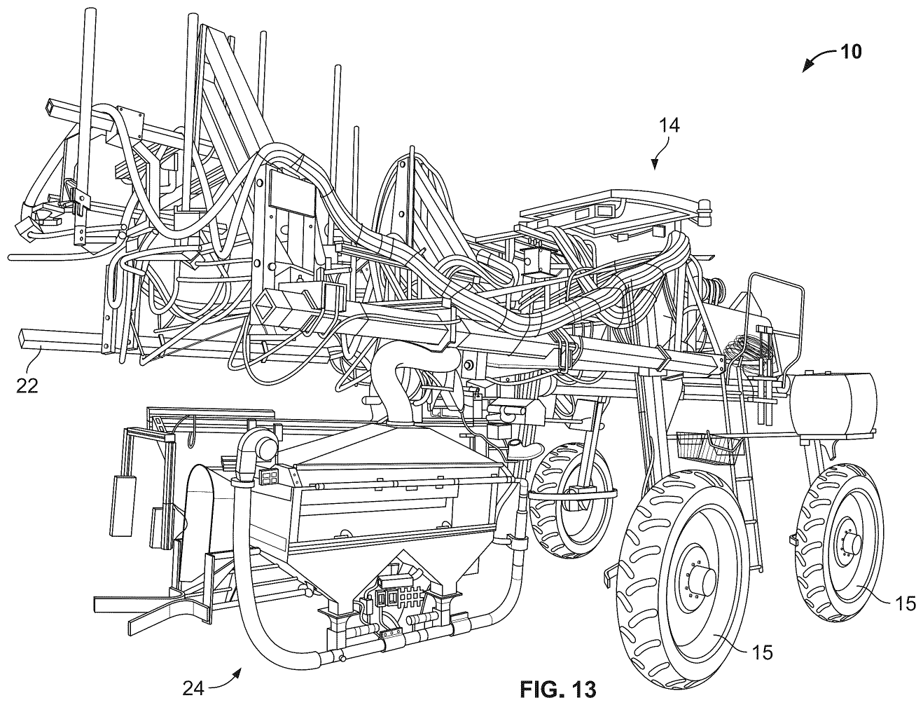

In addition, in the illustrated embodiment, the carriage 22 is configured to fold or collapse (FIG. 13) to allow for storage or transport of the pollination assembly 10 (e.g., from one field to another field, etc.). In particular in the illustrated embodiment, as shown in FIG. 13, end portions of the carriage 22 are configured to fold forward (relative to the tractor 14) to allow for storing the assembly 10 or transporting the assembly 10, as desired. In various examples, the end portions of the carriage 22 are configured to fold inward to a position in which the end portions of the carriage 22 are generally within or are spaced between left and right wheels 15 of the tractor 14 (and generally over or above (collection unit 24).

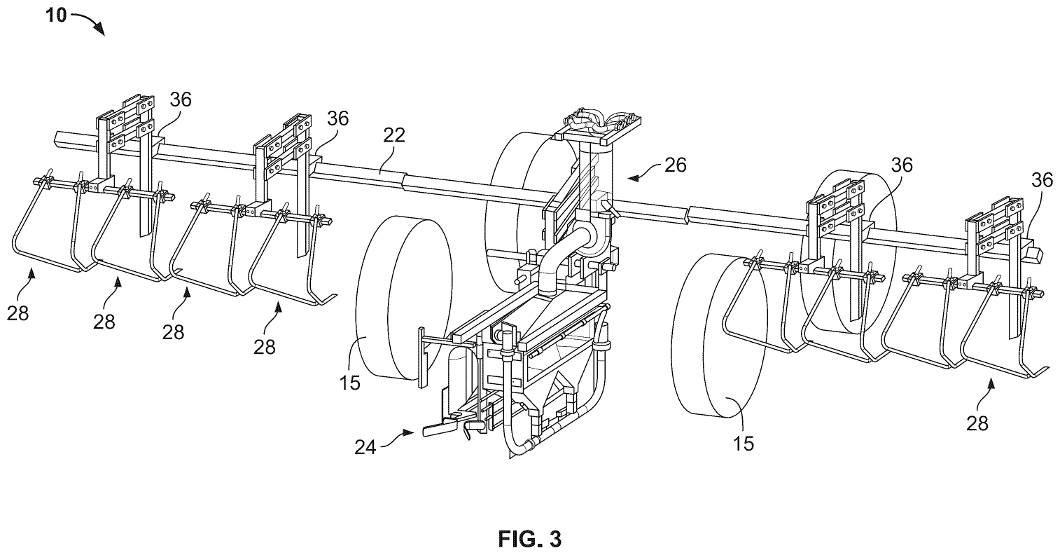

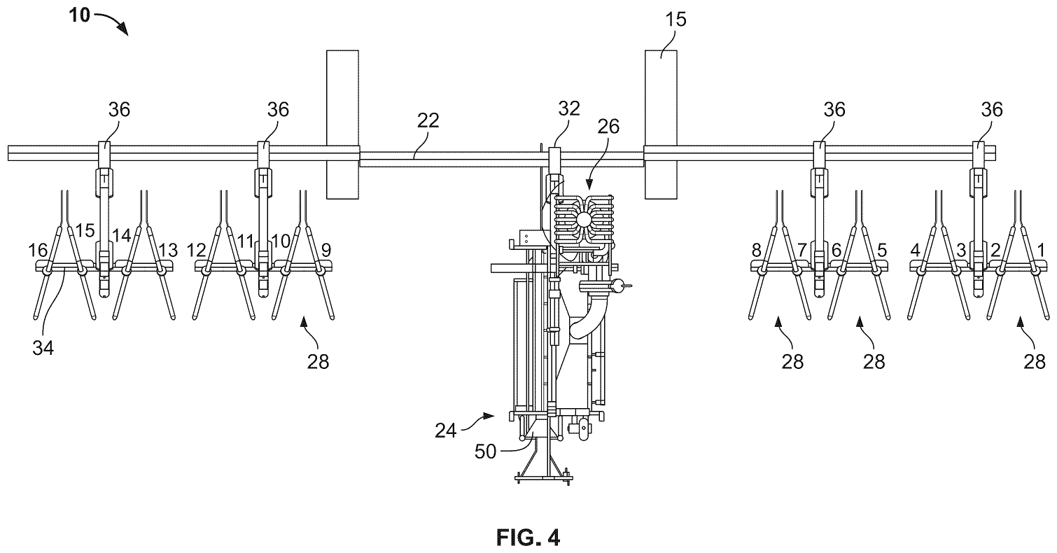

With additional reference to FIGS. 3-4, the pollination assembly 10 generally includes a collection unit 24, a distribution unit 26, and multiple applicator units 28 (or applicators) (e.g., at least one applicator unit 28, etc.), each of which is coupled to the carriage 22. As shown, the distribution unit 26 is positioned generally between (fluidically, etc.) the collection unit 24 and the applicator units 28. In this arrangement, the collection unit 24 is configured to release or remove (or displace or dislodge) pollen from the pollen-bearing plants 18 aligned with the collection unit 24, as the tractor 14 travels through the field 12. The distribution unit 26 is configured to then direct (and generally distribute (e.g., uniformly, etc.)) the released pollen from the collection unit 24 to each of the applicator units 28. And, the applicator units 28 are each configured to direct the received pollen, in a generally targeted manner, at the pollen-receiving plants 16 (in the rows of pollen-receiving plants 16 generally aligned with the corresponding applicator units 28). In particular, the applicator units 28 are each configured to direct the received pollen, in a targeted manner, at the flowers of the pollen-receiving plants 16. In this way, the pollen released (or displaced) from the pollen-bearing plants 18 is specifically delivered to (e.g., directed at, targeted at, aimed at, etc.) the flowers of the pollen-receiving plants 16, by the pollination assembly 10, to help facilitate pollination.

While one collection unit 24 and one distribution unit 26 are shown coupled to the carriage 22 (of the tractor 14) in FIGS. 1-3, it should be appreciated that an additional collection unit 24 and distribution unit 26 may also be coupled to the carriage 22 in other embodiments (e.g., generally adjacent the illustrated collection unit 24 (e.g., with both collection units (and corresponding distribution units) positioned between forward wheels 15 of the tractor 14, etc.), or generally spaced apart from the illustrated collection 24 with one or more of the applicator units 28 positioned therebetween, etc.). In this way, the tractor 14 may move through the field 12 and collect pollen from multiple rows of pollen-bearing plants 18 and direct (and deliver) the collected pollen to corresponding groups of pollen-receiving plants 16 via the applicator units 28.

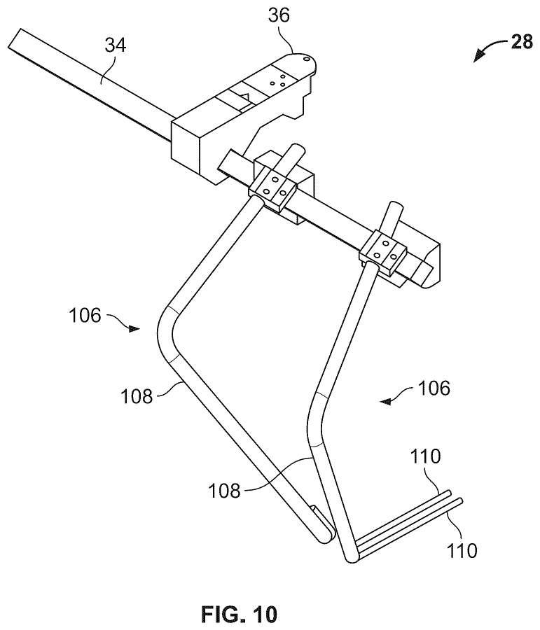

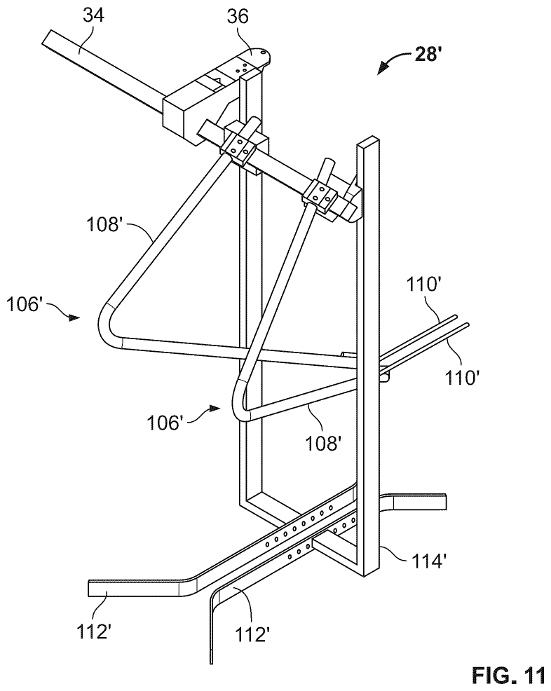

Referring now to FIGS. 3 and 4, the collection unit 24 and the distribution unit 26 are generally coupled together via a frame structure 30 (and corresponding frame members thereof). And, together, the collection unit 24 and the distribution unit 26 are coupled to the carriage 22 of the tractor 14 (via the frame structure 30) by a mount 32 (e.g., a lift, a boom, a support, etc.) (see, FIGS. 3-4). Additionally, each of the applicator units 28 similarly include a frame structure 34 (and corresponding frame members), and are also coupled to the carriage 22 of the tractor 14 (via the frame structure 34) by similar mounts 36 (see, also, FIGS. 10-11). The mounts 32, 36 are configured to support (and hold) the collection unit 24 and the distribution unit 26, as well as each of the applicator units 28, in desired positions relative to the carriage 22 (and tractor 14) and/or the plants 16, 18. In addition, the mounts 32, 36 may be actuated, as desired (e.g., individually, as a group, etc.), to move the units 24, 26, 28 to desired positions relative to the carriage 22 (and tractor 14) and/or relative to the plants 16, 18 in the field 12. For instance, the mounts 32, 36 may be actuated to raise or lower one or more of the collection and distribution units 24, 26 and/or applicator units 28 to properly position the units 24, 26, 28 relative to the plants 16, 18, depending on heights of the plants 16, 18, etc. Further, in some embodiments, such actuation of the mounts 32, 36 may be automatic, based on inputs received from one or more sensors coupled to the units 24, 26, 28, the carriage 22, and/or the tractor 14 (e.g., one or more sensors 38 configured for measuring, monitoring, etc. height of the plants as the tractor 14 moves through the field 12, heights and/or locations of tassels on the plants, heights and/or locations of silks on the plants, etc.).

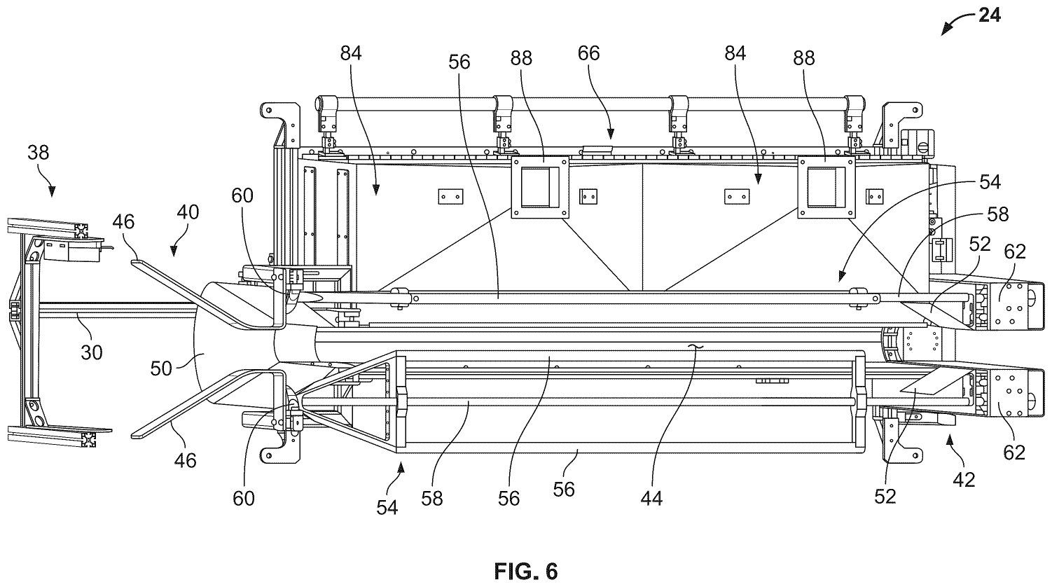

With additional reference to FIG. 6, the collection unit 24 of the pollination assembly 10 generally includes a forward guide assembly 40, a rearward guide assembly 42, and a channel 44 (or passage) extending generally between the forward guide assembly 40 and the rearward guide assembly 42 (e.g., generally longitudinally through the collection unit 24, etc.).

The forward guide assembly 40 is configured (e.g., oriented, sized, shaped, spaced, etc.) to receive and/or direct (e.g., sequentially, etc.) the pollen-bearing plants 18 (from one of the rows of pollen-bearing plants 18 aligned with the collection unit 24) into the collection unit 24, and in particular, into the channel 44 extending through the collection unit 24, as the tractor 14 moves the collection unit 24 through the field 12. In the illustrated embodiment, the forward guide assembly 40 includes a pair of arms 46 coupled to the frame structure 30, and extending generally forward of the collection unit 24 (e.g., in a forward direction of travel of the tractor 14 during use of the pollination assembly 10, etc.). The arms 46 are spaced apart (e.g., in a direction generally perpendicular to a longitudinal axis of the collection unit 24, etc.) to allow for movement of the pollen-bearing plants 18 between the arms (and into the channel 44). In addition, end portions of the arms 46 flare (or taper) generally outwardly, in order to gather, collect, funnel, etc. the pollen-bearing plants 18 between the arms 46 and into the channel 44 of the collection unit 24 (e.g., to accommodate variations in placement of pollen-bearing plants 18 in the rows while still facilitating receipt of the plants 18 within the channel 44, etc.). In addition, the forward guide assembly 40 includes a hood 50 positioned generally over the arms 46, to help direct and/or maintain the pollen-bearing plants 18 in the channel 44. As such, as the tractor 14 drives forward through the field 12, the arms 46 of the forward guide assembly 40 are generally aligned with one of the rows of the pollen-bearing plants 18. The flared end portions of the arms 46 then gather (and align) the plants 18 with the channel 44 of the collection unit 24, while the hood 50 helps ensure that the entire upper portion of each of the plants 18 in the row is received within the channel 44 (e.g., including the tassel, etc.). As the tractor 14 continues to drive forward, the pollen-bearing plants 18 from the given row are then received generally sequentially into the collection unit 24 (for removal of pollen therefrom).

The rearward guide assembly 42 is configured to direct the pollen-bearing plants 18 out of the collection unit 24, and in particular, out of the channel 44, after pollen is removed from the plants 18 (and as the tractor 14 continues to move forward through the field 12). In the illustrated embodiment, the rearward guide assembly 42 includes a pair of angled (or flared or tapered) tabs 52 coupled to the frame structure 30, and extending generally rearward of the collection unit 24 (e.g., in an opposite direction of travel of the tractor 14 during use of the pollination assembly 10, etc.). The tabs 52 (and more generally the rearward guide assembly 42), then, operate to inhibit the pollen-bearing plants 18 from becoming stuck within the channel 44, etc. (e.g., the tabs 52 operate to guide the pollen-bearing plants 18 out of the channel 44 and out of the collection unit 24, etc.).

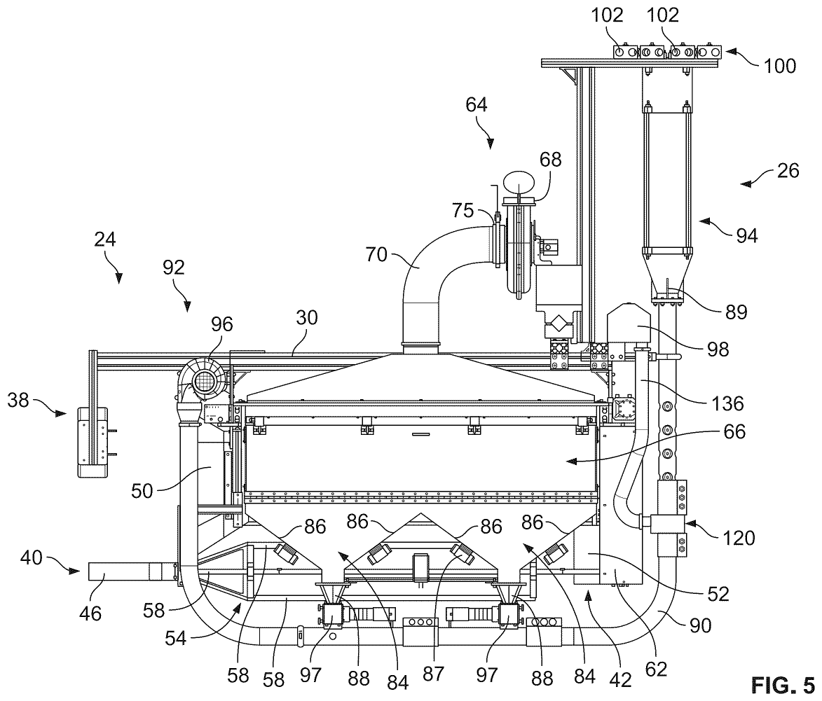

With continued reference to FIGS. 5-6, the collection unit 24 also includes agitators 54 associated with, or generally disposed within (or adjacent to), the channel 44. The agitators 54 are positioned generally in line with the guide assemblies 40, 42, and each is supported by the frame structure 30. In the illustrated embodiment, the collection unit 24 includes two agitators 54 disposed generally within (or adjacent to) the channel 44. In this arrangement, as the pollen-bearing plants 18 are received into the channel 44 of the collection unit 24, and move through the channel 44, the agitators 54 engage (e.g., operatively contact, etc.) the plants 18 (e.g., stalks of the plants 18, tassels of the plants 18, etc.) to facilitate release of pollen therefrom. In other embodiments, though, the collection unit 24 may include one agitator 54 or more than two agitators 54 (e.g., three agitators, four agitators, etc.) within the scope of the present disclosure.

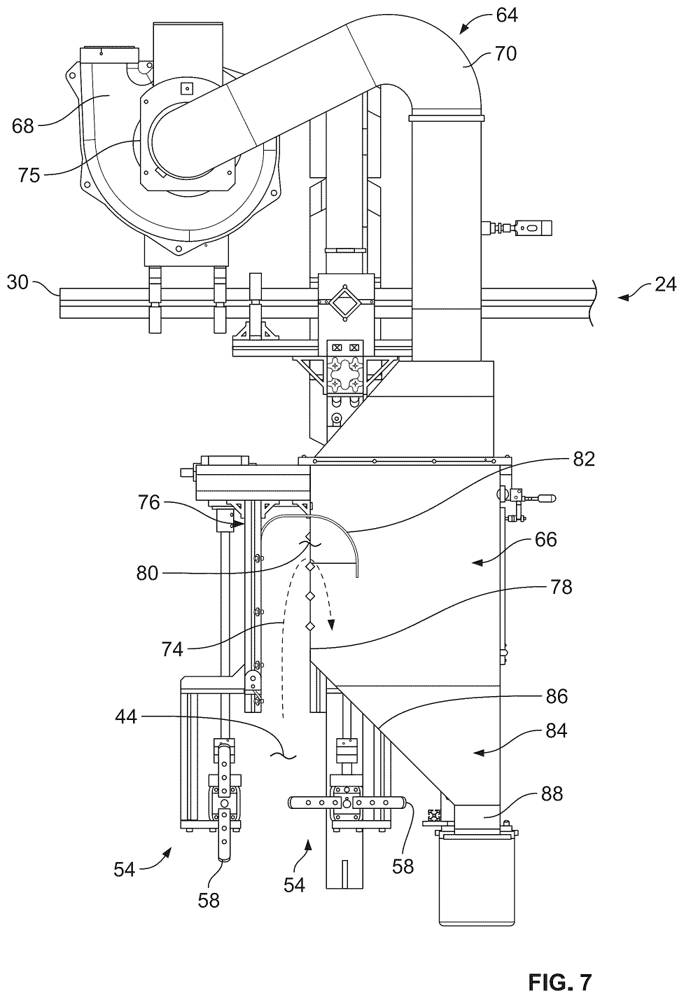

In the illustrated embodiment, each of the agitators 54 includes a pair of arms 56 coupled to a shaft 58 (e.g., a spindle, etc.) supported on (or by) the frame structure 30 via a forward bearing support 60 and a rearward drive 62 (e.g., a cogged belt drive/system, etc.). The arms 56, then, are configured to rotate via the shaft 58, through operation of the drive 62 to turn/rotate the shaft 58. In this example, the arms 56 of each of the agitators 54 are arranged to define a generally rectangular shape (see, FIG. 6). In addition, the arms 56 are spaced apart from each other by a desired distance (e.g., a distance between about 3 inches and about 9 inches, a distance of about 6 inches, etc.) to thereby provide an agitation zone in which the arms 56 contact the pollen-bearing plants 18. Further, the drives 62 of the collection unit 24 is configured to rotate the arms 56 of the corresponding one of the agitators 54 at desired speeds (e.g., so long as to inhibit damage to plants 18, etc.) (e.g., between about 100 rotations per minute (rpm) and about 500 rpm, at a speed of about 200 rpm, at a speed of about 300 rpm, at a speed of less than about 100 rpm, at a speed of greater than about 500 rpm, etc.), and in desired rotational directions (e.g., both in clockwise directions as viewed from a forward portion of the collection unit 24 (FIG. 7), both in counterclockwise directions as viewed from a forward portion of the collection unit 24 (FIG. 7), a left agitator 54 in a counterclockwise direction and a right agitator 54 in a clockwise direction as viewed from a forward portion of the collection unit 24 (FIG. 7), etc.). As such, the arms 56 are configured to engage each of the pollen-bearing plants 18 (e.g., within the agitation zone of each of the agitators, etc.) multiple times while the plants 18 are present in the channel 44 of the collection unit 24 to remove, dislodge, etc. pollen therefrom. It should be appreciated that the agitators 54 may include other configurations in other embodiments, for example, other arrangements of arms than illustrated (e.g., a single arm, three arms, four arms, more than four arms, arms arranged in other than rectangular configurations, etc.), configurations that do not include arms (e.g., chains, air jets, etc.), etc.

In operation of the pollination assembly 10, the agitators 54 (and in particular, the arms 56 of the illustrated agitators 54) are configured to engage upper portions of the pollen-bearing plants 18 (e.g., stalks of the plants 18, tassels of the plants 18, etc.) as the plants 18 pass into and through the channel 44 of the collection unit 24, to facilitate release of the pollen therefrom (e.g., from tassels of the plants 18, etc.). More generally, the agitators 54 are positioned to contact and/or disturb the male flowers and/or tassels of the pollen-bearing plants 18 to release the pollen from the plants 18.

In connection with the above, the illustrated collection unit 24 includes sensor 38 (e.g., camera, etc.) (e.g., at least one sensor, etc.) coupled to the frame structure 30 and positioned generally forward of the collection unit 24 (and forward of and generally higher than the guide assembly 40). In this embodiment, the sensor 38 is configured to sense, track, identify, etc. heights of the pollen-bearing plants 18 and/or locations of the male flowers and/or tassels of the pollen-bearing plants 18 (e.g., tassels of the pollen-bearing plants 18, etc.), for example, as the tractor 14 moves the pollination assembly 10 through the field 12 (and/or as the plants 18 approach and/or are received by the collection unit 24). In doing so, the sensor 38 may communicate the height of the pollen-bearing plants 18 with the collection unit 24, the tractor 14, etc. (e.g., with a computing device thereof, etc.) whereby (in response) the collection unit 24, tractor 14, etc. is configured to adjust (e.g., automatically, etc.) a height of the collection unit 24 and/or positions of the agitators 54 within the channel 44 to position the agitators 54 at a desired height to dislodge pollen from the pollen-bearing plants 18 as they pass through the collection unit 24. For instance, the collection unit 24, the tractor 14, etc. may actuate the mount 32 to adjust a height of the collection unit 24 (e.g., automatically, etc.) relative to the ground as desired, for example, based on input, communication, etc. from/by the sensor 38, etc. In this manner, the agitators 54 may be particularly positioned to generally align with the pollen-bearing plants 18 at certain heights to engage the stalks thereof and release pollen from the male-flowers and/or tassels of the plants 18 (e.g., to account for variations in the heights of the male flowers and/or tassels of the pollen-bearing plants 18, and/or to account for particular locations of the heads and/or male flowers of the pollen-bearing plants 18 as the tractor 14 approaches the plants 18 (e.g., depending on a type of the pollen-bearing plants 18, etc.), etc.).

With additional reference to FIG. 7, the pollination assembly 10 also includes an air system 64 (e.g., a first air system, etc.) coupled to (e.g., in fluidic communication with, etc.) the collection unit 24 for use in drawing pollen removed from the pollen-bearing plants 18 (by the agitators 54 within the channel 44) into a collection hopper 66 (or housing) of the collection unit 24. The air system 64 generally includes a blower 68 mounted on the frame structure 30 (of the collection unit 24) and coupled, via a conduit 70, to the collection hopper 66 (e.g., to an upper portion (or cover) of the collection hopper 66, etc.). The blower 68 is configured to generate/provide a vacuum within the collection hopper 66 (via the conduit 70) that operates to draw pollen from the channel 44 (as removed from the pollen-bearing plants 18 by the agitators 54) into the collection hopper 66 (as generally indicated by arrow 74 in FIG. 7). In example embodiments, the air system 64 (e.g., the blower 68, etc.) is configured to generate air flow (e.g., volumetric air flow, etc.) ranging from about 100 cubic feet per minute (cfm) to about 800 cfm (e.g., about 300 cfm, about 400 cfm, about 500 cfm, about 600 cfm, etc.). In connection therewith, a valve 75 may be provided at (or adjacent to), or included in, etc., the blower 68 to control (e.g., regulate, etc.) the air flow from the blower 68 that enters the conduit 70. In doing so, in some examples, the blower 68 may then provide, or cause, or facilitate, etc., a velocity of air entering into the collection hopper 66 (e.g., at opening 80 of the collection hopper 66, along path 74, etc.), from the channel 44, of between about 0.5 m/s and about 5 m/s (e.g., about 1 m/s, about 2 m/s, about 3 m/s, about 4 m/s, flow rates therebetween, or more or less, etc.).

In connection with the above, and as best shown in FIG. 7, the collection hopper 66 includes a pollen guide 76 (e.g., a slide, a baffle, etc.) configured to direct the pollen removed from the pollen-bearing plants 18, from the channel 44 to the collection hopper 66 (via the vacuum created by the blower 68 of the air system 64). In the illustrated embodiment, the pollen guide 76 is positioned along a sidewall 78 of the collection hopper 66 (adjacent the channel 44) and defines (and/or is associated with) opening 80 (within the sidewall 78) between the collection hopper 66 and the channel 44 (extending along a longitudinal length of the sidewall 78 (e.g., along an entire longitudinal length of the sidewall 78, along less than the entire longitudinal length of the sidewall 78, etc.)). And, an arcuate upper wall 82 (or baffle) of the pollen guide 76 extends through the sidewall 78 of the collection hopper 66. In this arrangement, the pollen guide 76 (and arcuate upper wall 82 thereof) operate to guide, direct, etc. the pollen removed from the pollen-bearing plants 18 from the channel 44, through the opening 80, and into the collection hopper 66.

In addition to the above, the collection hopper 66 includes recessed chambers 84 disposed along a bottom portion of the collection hopper 66. The chambers 84 are configured to collect the pollen removed from the pollen-bearing plants 18, as the pollen passes into the collection hopper 66 and settles from (or out of) the air flow/vacuum created by the air system 64. The chambers 84 include angled sides 86 configured to funnel the collected pollen to outlet ports 88 of the respective chambers 84, for subsequent transport to the distribution unit 26. While the illustrated collection hopper 66 includes two chambers 84, it should be appreciated that the collection hopper 66 may include only one chamber, or may include three chambers, or may include more than three chambers in other example embodiments. In addition, in some example embodiments, the collection hopper 66 may include containers coupled to the outlet ports 88 of the chambers 84, whereby the pollen that settles into the chambers 84 may instead be collected within the containers (instead of transported to the distribution unit 26).

In connection with the above, in the illustrated embodiment, the pollen is configured to settle in the collection hopper 66 (within the chambers 84), from the air flow/vacuum created by the air system 64, based on both deceleration of the pollen (and air flow) within the collection hopper 66 and inertia. For instance, with regard to deceleration, the collection hopper 66 has a cross-sectional area that is larger than that generally defined by the upper wall 82 where the pollen enters the collection hopper 66. As such, the pollen and air decelerate as they enter the collection hopper 66. And, once the air velocity reaches (e.g., slows to, etc.) a threshold value (e.g., about 0.5 m/s, about 1 m/s, about 1.5 m/s, velocities therebetween, etc.) (e.g., based on a size and/or type of the pollen being collected, etc.), the air can no longer hold/transport the pollen within the air and the pollen falls into one of the chambers 84 (e.g., onto a side 86 of one of the chambers 84, to an outlet port 88 of one of the chambers 84, etc.). And, with regard to inertia, as the pollen enters the collection hopper 66 (via the opening 80), inertia forces the pollen to ride along the surface (e.g., inner or lower surface, etc.) of the upper wall 82, which in turn directs (or guides) the pollen (or puts the pollen on a trajectory) generally toward the chambers 84 of the collection hopper 66 (e.g., downward toward a bottom portion of the collection hopper 66, etc.). That said, it should be appreciated that in some embodiments, only inertia may be used to separate the pollen from the air flow in the collection hopper 66 (e.g., without use of deceleration of the air flow, etc.).

As best shown in FIG. 5, the collection unit 24 is coupled to the distribution unit 26 by a conduit 90. In connection therewith, the pollination assembly 10 includes another air system 92 (e.g., a second air system, etc.) coupled to the conduit 90 (broadly, coupled to the collection unit 24, the distribution unit 26 and the applicator units 28) (e.g., in fluidic communication therewith, etc.) and configured to move the settled pollen from the outlet ports 88 of the collection hopper 66 to a distributor 94 of the distribution unit 26 (and then on to the applicator units 28). In the illustrated embodiment, the air system 92 includes first and second blowers 96, 98 mounted on the frame structure 30 and coupled in fluid communication with the conduit 90. In this arrangement, the blowers 96, 98 are configured (e.g., selectively operated individually, together, etc.) to generate/provide an air flow within the conduit 90 that operates to direct (e.g., push, carry, etc.) the pollen from the outlet ports 88 of the collection hopper 66 to the distributor 94. As noted, the blowers 96, 98 may be operated together to transport the pollen from the outlet ports 88 of the collection hopper 66 to the distributor 94, or the blowers 96, 98 may be operated individually (e.g., only the blower 96 may be operated, or only the blower 98 may be operated, etc.).

In connection with operation of the blower 96, air locks 97 (broadly, closures) are provided at (e.g., coupled to, etc.) the outlet ports 88. The air locks 97 are configured to actuate and selectively allow pollen to flow through the outlet ports 88 and to the conduit 90. In doing so, the air locks 97 are configured to separate the air flow and pressure within the chambers 84 (of the collection hopper 66) from the conduit 90. This allows for independent adjustment of air flow, from the blower 96, for example, for conveying (e.g., pushing, etc.) the pollen through the conduit 90 and into the distributor 94. Here, since the conduit 90 is generally air tight, positive pressure from the blower 96 can be used to convey (e.g., push, etc.) the pollen through the conduit 90 (and to the distributor 94).

And, in connection with operation of the blower 98, the air locks 97 may or may not be included at the outlet ports 88 of the collection hopper 66. Here, the blower 98 is configured to draw the pollen (e.g., via a vacuum generated at the chambers 84, at the outlet ports 88, etc.) from the chambers 84, through the outlet ports 88, and into the conduit 90. The blower 98 is then configured to direct the pollen through the conduit 90 and to the distributor 94.

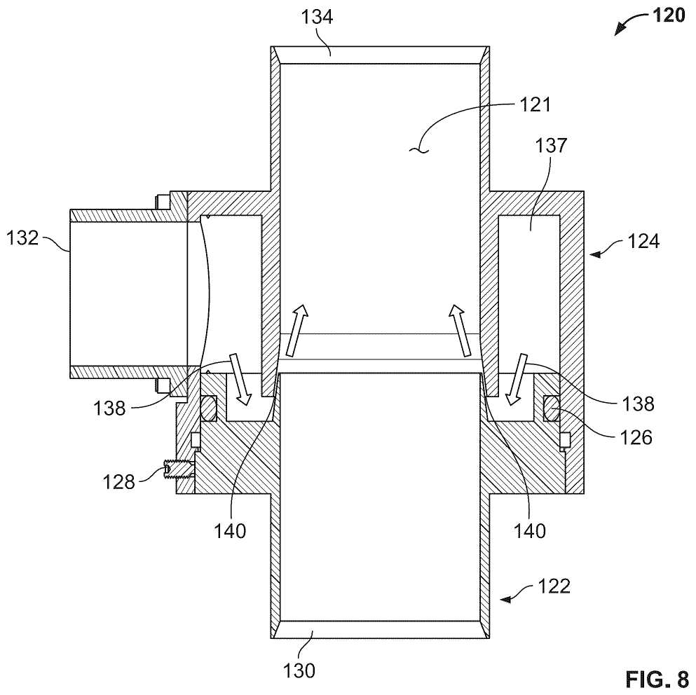

As part of this operation of the blower 98 of the air system 92, to direct the pollen to the distributor 94, an air conveyor 120 (e.g., as part of the air system 92, etc.) is coupled to the conduit 90, generally between the blower 98 and the conduit 90, to facilitate the desired air flow in the conduit 90 (e.g., the vacuum at the outlet ports 88 of the chambers 84, etc.). As shown in FIG. 8, the air conveyor 120 generally includes a body 122 and an annular air plenum 124 coupled to the body 122 (e.g., via a threaded connection, an O-ring 126, and a set screw 128; etc.). The body 122 includes an inlet portion 130 configured to couple inline to the conduit 90. And, the air plenum 124 includes an inlet portion 132 configured to couple to the blower 98 (via conduit 136 (FIG. 5)), and an outlet portion 134 configured to couple inline to the conduit 90 (on the distributor 94 side of the air conveyor 120). In the illustrated embodiment, the outlet portion 134 of the air plenum 124 defines a generally constant diameter along its length. In addition in this embodiment, the inlet portion 130 of the body 122 defines a generally constant diameter along its length, which is also generally the same as the diameter defined by the outlet portion 134 of the air plenum 124. As such, a channel 121 extending through the air conveyor 120 (from the inlet portion 130 of the body 122 to the outlet portion 134 of the air plenum 124) defines a generally constant diameter extending along a length of the channel 121.

In this arrangement, the air conveyor 120 is configured to receive pressured air from the blower 98, at the inlet portion 132, and direct the air generally circumferentially around the air plenum 124 (via channel 137 extending circumferentially around the plenum 124) and direct the air into the outlet portion 134 of the air plenum 124, via an air gap (or discharge) 140 (as generally indicated by the arrows 138 in FIG. 8). In the illustrated embodiment, the air gap 140 is defined between the body 122 and the air plenum 124 and extends generally circumferentially around the air conveyor 120. In other embodiments, though, the air gap 140 may define slotted openings arranged generally circumferentially around the air conveyor 120, or in still other embodiments, the air gap 140 may extend only partially around the air conveyor 120. Further, in some embodiments, the air gap 140 may be disposed generally within the outlet portion 134 and arranged to direct air into the outlet portion 134 in a manner that is generally parallel to sidewalls of the outlet portion 134, etc.

The air conveyor 120 is configured, via the air gap (or discharge) 140, to generally convert the pressurized air from the blower 98 into a relatively high velocity flow of air (with relatively low pressure) within the outlet portion 134 of the air plenum 124. And, in turn, the high velocity air flow in the outlet portion 134 of the air plenum 124 generates negative pressure at the inlet portion 130 of the body 122, which generates negative pressure along the conduit 90 at the chambers 84 of the collection hopper 66. In the illustrated embodiment, the air plenum 124 of the air conveyor 120 is adjustable (e.g., via a threaded connection therebetween, etc.) relative to the body 122. This allows for adjusting a size of the air gap (or discharge) 140, for example, to adjust air flow, suction pressure, air consumption, and required air pressure, etc. within the conduit 90, as desired. In addition in the illustrated embodiment (again, as generally illustrated by the arrows 138 in FIG. 8), the air gap 140 is configured to direct the higher velocity air toward a center or middle region (or portion) of the outlet portion 134 of the air plenum 124 (and away from sidewalls of the outlet portion 134, etc.). In doing so, the air flow generally detaches from the sidewalls of the outlet portion 134 and creates a momentum of air towards the distributor 94 (and, in turn, potentially helping to generate the negative pressure at the inlet portion 130 of the body 122, which generates negative pressure along the conduit 90 at the chambers 84 of the collection hopper 66).

In example embodiments, the air system 92 (e.g., the blower 96, the blower 98, the blowers 96, 98, etc.) is configured to generate air flow ranging from about 10 cfm to about 100 cfm (e.g., about 30 cfm, about 40 cfm, about 50 cfm, about 60 cfm, etc.). In general, in such embodiments, the air flow generated by the air system 92 to transport the collected pollen to the distributor 94 (and ultimately to the applicator units 28) is less than the air flow generated by the air system 64 to collect the pollen (within the collection hopper 66) removed from the pollen-bearing plants 18 (e.g., a ratio of air flow generated by the air system 64 to air flow generated by air system 92 is about 5:1, about 10:1, about 100:1, ratios therebetween, or more or less, etc.).

With continued reference to FIG. 5, the distribution unit 26 includes the distributor 94, and a manifold 100 coupled to the distributor 94. The distributor 94 is configured to reduce the flow of the pollen (e.g., slow the air flow carrying the pollen, etc.) received from the collection hopper 66 and then deliver the pollen to the manifold 100. In particular, the pollen from the collection hopper 66 is introduced into the distributor 94 at a lower portion of the distributor 94. The air flow from the air system 92 then pushes, or carries, the pollen upward through the distributor 94. In doing so, the distributor 94 slows the flow of the pollen therein and then discharges the pollen, from an upper portion of the distributor 94, to the manifold 100 (for distribution to the applicator units 28). In addition, in some examples, the distributor 94 may also help facilitate mixing of the pollen, etc. prior to delivery of the pollen to the manifold 100.

In particular in the illustrated embodiment, the distributor 94 is configured to decelerate the pollen (e.g., to a threshold velocity within the distributor 94 (e.g., about 0.5 m/s, about 1 m/s, about 1.5 m/s, velocities therebetween, etc.), etc.), as the pollen is received therein from the conduit 90, prior to transferring the pollen to the manifold 100 and prior to the manifold 100 distributing the pollen to the applicator units 28. In decelerating the pollen, the distributor 94 also generally mixes the pollen therein to provide a generally even distribution of pollen in preparation for delivering the pollen to the manifold 100. The distributor 94 is configured to then discharge the generally uniform and mixed pollen, at the decelerated velocity, to the manifold 100 and each of ports 102 thereof (again, in a generally even distribution of the pollen). In this way, the air flow carrying the pollen is controlled within the pollination assembly 10 (e.g., via the distribution unit, etc.), so that a generally consistent and/or uniform and/or even distribution (and delivery) of pollen is provided to the manifold 100 and, in turn, to each of the applicator units 24 via the manifold 100 (and ports 102) (e.g., within about ten percent or less, etc.). In other words, the distributor 94 is configured to distribute the pollen generally evenly within the airflow so that the single stream of pollen coming from the collection unit 24 may be subsequently divided (or split), at the manifold 100, into outgoing streams that all have about equal amounts of pollen. This allows for pollen collected from the single (or double) row of pollen-bearing plants 18 to be distributed generally evenly to each of the multiple applicator units 28 and the multiple rows of pollen-receiving plants 16 aligned therewith.

In the illustrated embodiment, the distributor 94 includes a generally vertical column to facilitate decelerating the pollen as it enters the distributor 94 and to facilitate mixing of the pollen prior to delivery to the manifold 100. In addition, in example embodiments, a diameter of the distributor 94 (e.g., the column, etc.) may increase from the lower portion of the distributor 94 to the upper portion of the distributor 94 (e.g., from about three inches at the lower portion of the distributor 94 to about eight inches at the upper portion of the distributor 94, etc.). This configuration of the distributor 94, with the increasing diameter, further helps to decelerate the pollen (and reduce the air flow carrying the pollen) as it enters the distributor 94 and to mix the pollen prior to distribution of the pollen to the manifold 100.

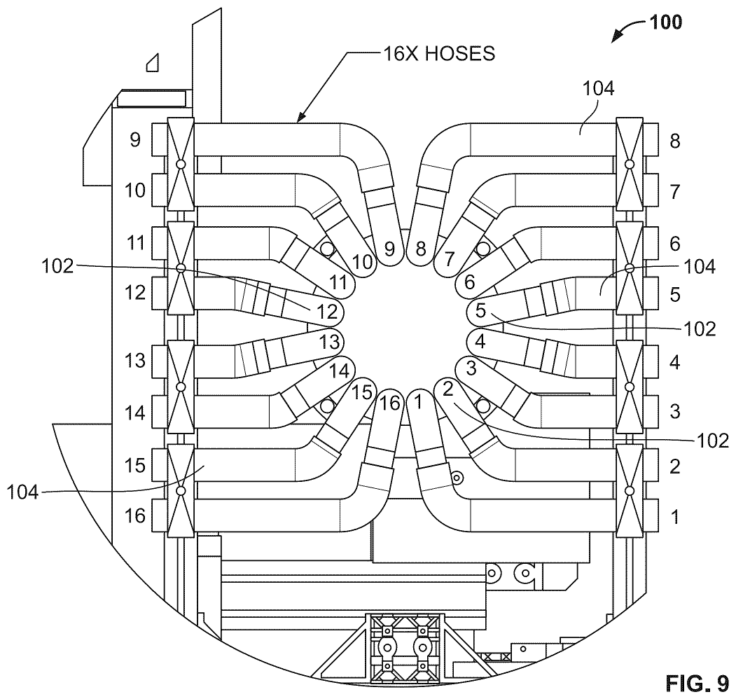

With additional reference to FIG. 9, the manifold 100 includes multiple ports 102, each coupled to one of the applicator units 28 via a conduit 104. In the illustrated embodiment, the manifold includes sixteen ports 102, each associated with a corresponding one of the applicator units 28 (e.g., corresponding to the numbering in FIG. 4, etc.). The air system 92, in turn, is configured to further transport the pollen delivered to the manifold 100 (from the distributor 94) to the applicator units 28. In doing so, the pollen is discharged from the applicator units 28 at a desired rate to facilitate targeted delivery of the pollen to the flowers of the pollen-receiving plants 16. That said, in example embodiments, the air system 92 may be configured to generate an air flow at each of the applicator units 28 (e.g., at nozzles of the applicator units 28, etc.) having an air speed of between about 1 m/s and about 10 m/s (e.g., about 1 m/s, 2 m/s, about 3 m/s, about 4 m/s, about 5 m/s, about 6 m/s, about 7 m/s about 8 m/s, about 9 m/s, etc.), for instance, to generate and/or produce a desired blowing distance/rate of the pollen toward the pollen-receiving plants 16. In addition (or alternatively), in example embodiments, the air system 92 may be configured to generate a generally uniform air flow across all of the applicator units 28 (via the manifold 100) so that the air flow at each of the applicator units 28 is within about twenty percent (or more or less) of each other.

Referring now to FIGS. 4 and 10, the applicator units 28 each include two applicators 106 mounted to the frame structure 34 (of the applicator units), which in in turn is coupled to the carriage 22 by the mount 36 (as described above). The applicator units 28 are configured to receive the mixed pollen from the distributor 94 (via the manifold 100 and conduit 104) and apply the pollen to the pollen-receiving plants 16 (in the rows of plants 16 in the field 12 aligned with the applicator units 28).

In the illustrated embodiment, the applicators 106 of the applicator units 28 each include a tube 108 (e.g., a drop tube, etc.) and a nozzle 110 coupled to an end portion of the tube 108. In addition, the tubes 108 of the applicators 106, of each applicator unit 28, are arranged relative to each other so as to define a guide for directing, funneling, channeling, etc. the pollen-receiving plants 16 (in the row aligned with the applicator unit 28) generally between the two applicators 106 (e.g., into a pollination zone or into a pollination window (e.g., into a correct or proper position, etc.) for receiving pollen discharged by the nozzles 110, etc.). For example, forward portions 108a of the tubes 108 of the applicators 106 are generally spaced apart to accommodate, facilitate, etc. movement of the pollen-receiving plants 16 therebetween. The tubes 108 then angle generally inward (or toward each other) so that the nozzles 110 of the applicators 106 are positioned adjacent each other. Further, the nozzles 110 are generally directed inwardly toward each other (and at the pollen-receiving plants 16 passing between the nozzles 110). In this arrangement, then, the nozzles 110 are configured to provide pollen generally directly at the pollen-receiving plants 16 (in a targeted manner, as a directed discharge, etc.) as the plants pass through the applicator unit 28 (and between the nozzles 110) (e.g., generally targeted at the flowers of the plants 16, etc.).

Also in the illustrated embodiment, the applicator units 28 are adjustable to accommodate different heights of the pollen-receiving plants 16 passing through the applicator units 28, and/or different heights/locations of flowers on the pollen-receiving plants 16. For instance, heights of the pollen-receiving plants 16 may be determined by one or more sensors (e.g., sensor 38, etc.). In connection therewith, the sensor(s) may communicate the height of the pollen-receiving plants 16 to the tractor 14, etc. (e.g., a computing device thereof, etc.) whereby (in response) the tractor 14, etc. is configured to adjust (e.g., automatically, etc.) heights of the applicator units 28, via the mounts 36, as needed, to locate the nozzles 110 at desired heights relative to the pollen-receiving plants 16 and/or flowers thereof (e.g., about 6 inches or more or less above the flowers of the pollen-receiving plants, etc.). In addition, the tubes 108 of the applicator units 28 may be pivotally coupled to the frame structure 34, to allow the tubes 108 to rotate (or pivot) relative to the frame structure 34 and move the nozzles 110 away from each other and toward each other, as needed or desired, to facilitate contact of the nozzles 110, for example, with the pollen-receiving plants 16 passing therebetween, and targeted discharge of the pollen at/to the flowers of the pollen-receiving plants 16. In this way, the positions of the nozzles 110 of the applicator units 28 are adjustable, and are able to be specifically positioned relative to the pollen-receiving plants (e.g., above the flowers of the pollen-receiving plants 16 by a desired distance, etc.) to facilitate the targeted distribution of pollen to the flowers.

In some example embodiments, the pollination assembly 10 may include one or more sensors configured to measure viability of the pollen (e.g., NIR sensors, etc.), configured to measure flow of the pollen (e.g. mass flow sensors, etc.), etc. as the pollen is received into and/or as the pollen flows through the assembly 10 (e.g., sensors 87, 89, etc.). For instance, in one example embodiment, where the sensor is configured to measure viability of the pollen, the sensor may be installed at the collection hopper 66 of the collection unit 24. In doing so, as pollen is received into the hopper 66, some of the pollen may be collected and evaluated via the sensor (e.g., at location/sensor 87 in FIG. 5 along side 86, etc.). In another example, where the sensor is configured to measure viability and/or flow, the sensor may be placed in a flow path of the pollen, for instance, at a base of the distributor unit 94 and after the air amplifier 120 (see, sensor 89, etc.). That said, in still other examples, the sensor may be located at any inline position after the collection unit output ports 88 and before reaching the manifold 100.

In example embodiments, where the sensor is configured to measure pollen viability, a MircoNIR sensor (e.g., from VIAVI Solutions Inc., etc.) may be used to measure the viability of the pollen received in the assembly 10. In such example embodiments, the sensor is configured to measure pollen moisture that correlates to fresh pollen quality. This information may be transferred to an operator or computing device, and used to determine when to stop operating as pollen quality decreases. In connection therewith, NIR spectra may be interpreted to determine how much water is contained in plant samples such as pollen. High pollen moisture content may be highly correlated to high fresh pollen germination, and may be a good measure of pollen quality. In some example embodiments, the NIR sensor may also be used to observe a reduction in pollen moisture content which may be indicative of the closing of the effective natural pollination window.

In example embodiments, where the sensor is configured to measure flow of pollen (e.g., a mass of pollen, etc.) through the pollination assembly 10 (e.g., a mass flow sensor, etc.), the mass flow may be used to make adjustments to the rates of the air handling system and speed of the tractor to insure efficient operation (e.g., optimized operation, etc.). In addition, one or more pollen yield maps may be generated based on the measured flow. And, in turn, the pollen yield maps may be correlated to seed yield maps and the NIR sensor viability information to create future yield prediction maps. Further, such mass flow may be used to determine pollen variability within a field, to determine pollen health, as part of historical data comparison, to assist in harvest planning, for research and development, for economic analysis, etc.

FIG. 11 illustrates an example embodiment of another applicator unit 28′ that may be used with the pollination assembly 10. In this example embodiment, the applicator unit 28′ again includes two applicators 106′, each comprising a tube 108′ and a nozzle 110′ coupled to an end portion of the tube 108′. In connection therewith, the applicator unit 28′ (and nozzles 110′) operate in substantially the same manner as described above for the applicator units 28. Additionally in this embodiment, the applicator unit 28′ includes a pair of guides 112′ mounted to a frame 114′ (which in turn is mounted to the frame structure 34 and mount 36). The guides 112′ define a channel generally aligned with (and generally below) the applicators 106′, and are configured to direct the pollen-receiving plants 16 into the channel. In doing so, the pollen-receiving plants 16 are generally aligned and directed to pass through the channel and between the nozzles 110′ of the applicators 106′.

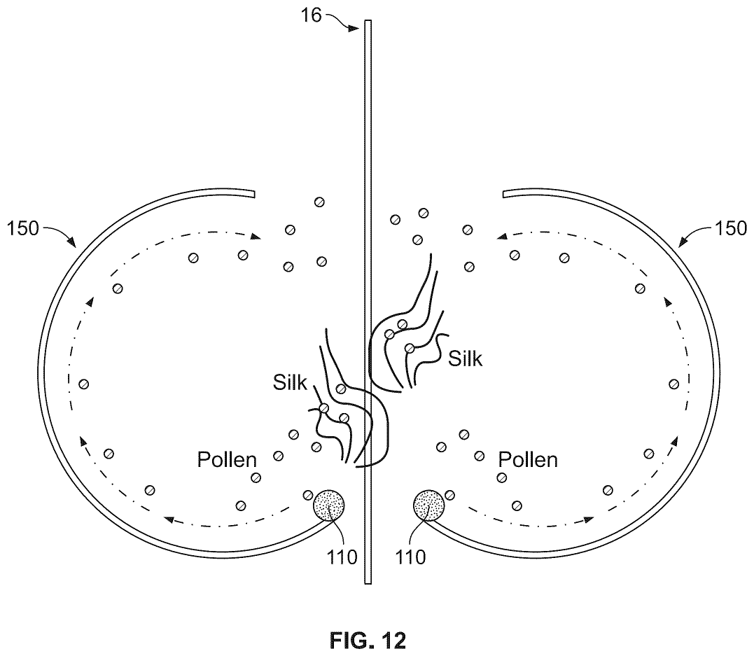

FIG. 12 illustrates an example embodiment of a recirculation duct 150 that may be included in each of the applicator units 28 (or applicator units 28′) of the pollination assembly 10. In particular, the duct 150 may be coupled to each nozzle 110 of the applicator units 28, and may extend generally upward and outward relative to the nozzle 110. In addition, the duct 150 may extend a distance longitudinally along the nozzle 110 and/or a distance longitudinally rearward (and way from) the nozzle 110. In the illustrated embodiment, the duct 150 defines a generally rounded configuration, but may have other configurations in other embodiments.

In operation, then, as the applicator units 28 discharge, direct, etc. pollen at pollen-receiving plants 16 passing between the nozzles 110 of the applicator units 28, the duct 150 coupled to each of the nozzles 110 is configured to contain the pollen that does not contact the pollen-receiving plants and redirect the pollen back at the pollen-receiving plants 16 (e.g., the silks of the pollen-receiving plants 16, etc.). In this way, a higher percentage of pollen discharged by the nozzles 110 of the applicator units 28 may be directed at and/or may contact the pollen-receiving plants 16.

In example embodiments, the pollination assembly 10 may include additional air systems (e.g., third air systems, fourth air systems, fifth air systems, or more, etc.), and additional air conveyors and distributors associated with the air systems, to control flow of air (and pollen) from the collection unit 24 to the applicator units 28 (or to storage, etc.). In doing so, the additional air systems (and corresponding distributors and air conveyors) may be used in sequence to decelerate (e.g., iteratively, etc.) and/or accelerate (e.g., iteratively, etc.) the air flow carrying the pollen, from the collection unit 24, until a desired flow is achieved (e.g., in generally the same manner as described above with regard to operation of the air system 96 (and blower 98), air conveyor 120, and distributor 90, etc.). In this way, in some examples, a relatively high air flow may be used to collect the pollen at the collection unit 24, while the additional air systems (and air conveyors and distributors) may be used to decelerate the air flow (carrying the pollen) to provide a relatively low air flow to direct the pollen to the applicator units 28, or to otherwise process the pollen (e.g., combine additives with the pollen, collect the pollen, etc.). In addition, in some examples, the additional air systems (and air conveyors and distributors) may be used to accelerate the air flow carrying the pollen, as needed, for instance at one or more of the applicator units 28, to help ensure that desired flow rates of pollen (e.g., for discharge, etc.) are achieved at the applicator units 28 (e.g., where an air flow carrying the pollen has decelerated too much, the additional air systems (and air conveyors and distributors) may be used adjacent one or more of the applicator units 28 to accelerate the air flow to a desired rate for discharge by the applicator units 28; etc.).

In other words, each of the multiple air systems, and corresponding air conveyors and distributors associated with the air systems, may be used to decelerate the collected pollen, in a step-wise manner (per air system, air conveyor, and distributor), to achieve a desired air flow of the collected pollen, based on desired final processing of the pollen (and/or specific use cases for the pollen) in/by the pollination assembly 10 (be it subsequent application to the pollen-receiving plants 16, collection, storage, etc.). For instance, in one example embodiment, the second air system 92 (and blower 98), air conveyor 120, and distributor 94 may be configured to decelerate an air flow of the pollen collected by the collection unit 24 about ten times. Then, a third air system, air conveyor, and distributor arranged in series with the second air system 92 (and blower 98), air conveyor 120, and distributor 94 may be configured to decelerate the air flow of the pollen received from the distributor 94 another about ten times (such that the overall air flow generated by the air system 64, carrying the collected pollen from the collection unit 24, is reduced about one-hundred times). In some example embodiments, multiple air systems, distributors and air conveyors may be arranged in series between the collection unit 24 and the manifold 100. In some example embodiments, air systems, distributors and air conveyors may additionally (or alternatively) be arranged in series between the manifold 100 and the applicator units 28.

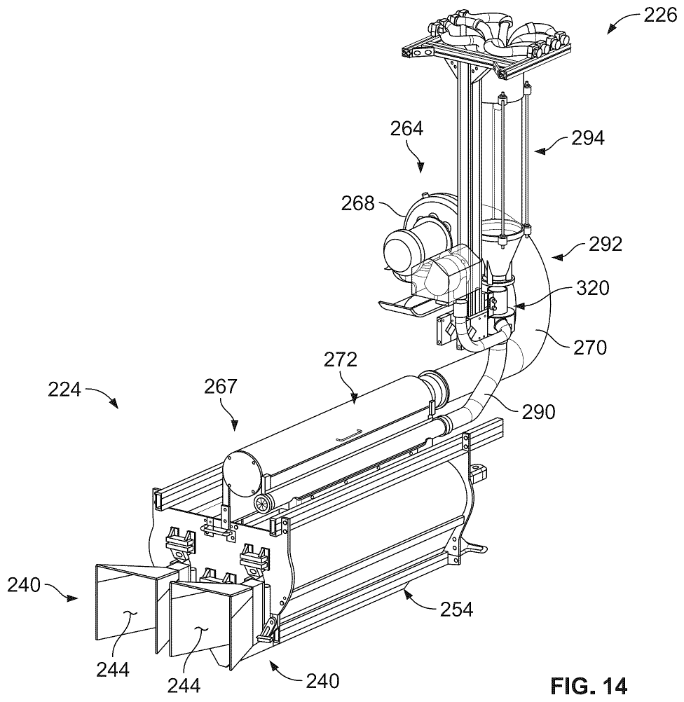

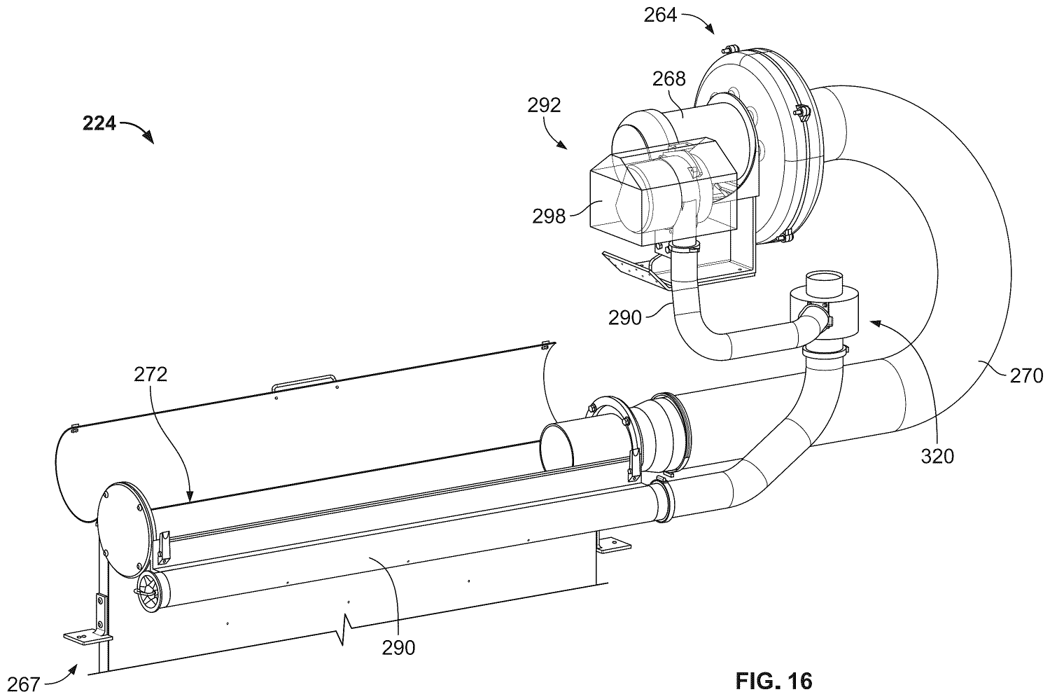

FIGS. 14-18 illustrate another example embodiment of a collection unit 224 and a distribution unit 226 that may be used in the pollination assembly 10 of FIGS. 1-13. In particular, the collection unit 224 and the distribution unit 226 of this embodiment may be coupled to the carriage 22 (of the tractor 14) in place of the collection unit 24 and the distribution unit 26, whereby the pollination assembly 10 may again be used to direct, move, deliver, convey, transfer, etc. pollen between plants, for example, for effecting pollination, etc. of the plants (as generally described above). In particular, the collection unit 224 is configured to release or remove (or displace or dislodge) pollen from the pollen-bearing plants 18 aligned with the collection unit 224, as the tractor 14 travels through the field 12. And, the distribution unit 226 is configured to then direct (and generally distribute (e.g., uniformly, etc.)) the released pollen to each of the applicator units 28 (which are configured to operate as described above).

As shown in FIG. 14, the collection unit 224 and the distribution unit 226 are generally coupled together, for example, via a frame structure (e.g., frame structure 30 as generally described above for the collection unit 24 and the distribution unit 26, etc.). And, together, the collection unit 224 and the distribution unit 226 may be coupled to the carriage 22 of the tractor 14 (via the frame structure 30) by mount 32 (again, as generally described above for the collection unit 24 and the distribution unit 26).

The collection unit 224 generally includes forward guide assemblies 240 and channels 244 (or passages) extending generally from the respective ones of the forward guide assemblies 240 and longitudinally through the collection unit 224. The forward guide assemblies 240 are each configured (e.g., oriented, sized, shaped, spaced, etc.) to receive and/or direct (e.g., sequentially, etc.) pollen-bearing plants into the collection unit 224, and in particular, into the corresponding channels 244 extending through the collection unit 224, as the tractor 14 moves the collection unit 224 through a field. While two guide assemblies 240 (and corresponding channels 244) are illustrated in FIG. 14, it should be appreciated that the collection unit 224 may include only one guide assembly (and channel) in other embodiments, or more than two guide assemblies (and channels) in still other embodiments.

The illustrated collection unit 224 also includes agitators 254 associated with, or generally disposed within (or adjacent to), each of the channels 244 (only one agitator 254 is visible in FIG. 14). The agitators 254 are substantially the same as the agitators 54 described above for the collection unit 24, and are configured to operate in substantially the same manner (to remove pollen from pollen-bearing plants as the plants enter the channels 244 of the collection unit 224). In addition in this example embodiment, air system 264 (e.g., a first air system, etc.) and air system 292 (e.g., a second air system, etc.) are both coupled to (e.g., in fluidic communication with, etc.) the collection unit 224. In connection therewith, the air system 264 is configured to draw pollen removed from pollen-bearing plants (by the agitators 254 within the channel 244) into a collector 267 (e.g., a hopper-less collector, etc.) of the collection unit 224, and then, the air system 292 is configured to draw the collected pollen out of the collector 267 and direct the pollen to distributor 294.

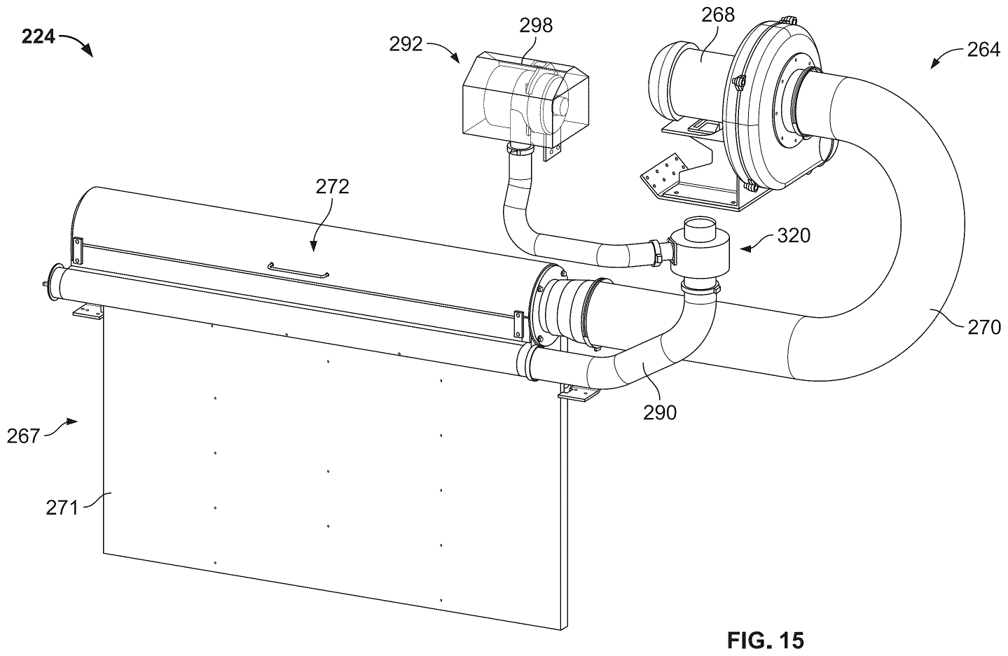

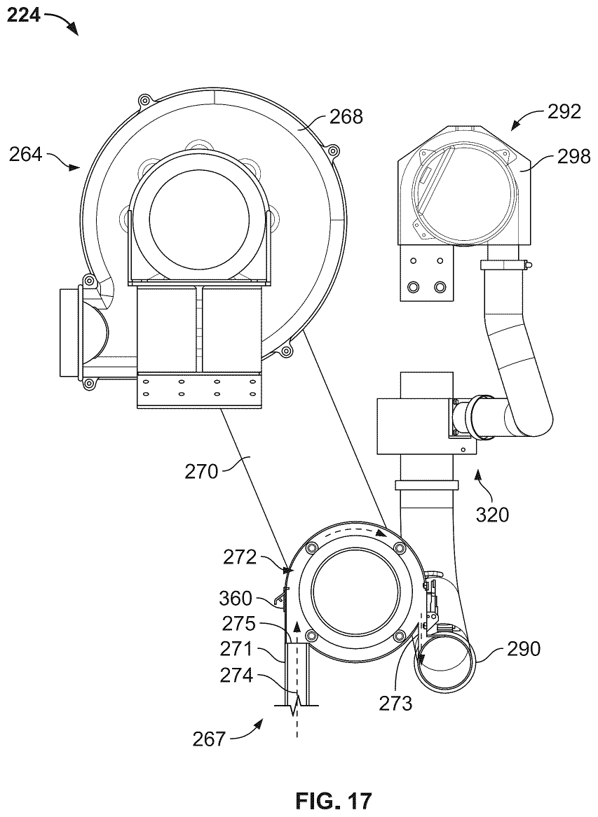

With reference to FIGS. 15-17, the air system 264 generally includes a blower 268 coupled, via a conduit 270, to the collector 267 (e.g., to an end portion of a separation chamber 272 of the collector 267, etc.). The blower 268 is configured to generate/provide a vacuum within the collector 267 (via the conduit 270) that operates to draw pollen from each of the channels 244 (as removed from the pollen-bearing plants by the agitators 254) and into the separation chamber 272 of the collector 267 (as generally indicated by arrow 274 in FIG. 17). The air system 264 (e.g., the blower 268, etc.) is configured to generate air flow (e.g., volumetric air flow, etc.) in the same manner as generally described herein with reference to the air system 64. Similarly, the air system 292 includes a blower 298 coupled, via conduit 290 (e.g., a trunk line, etc.), to the collector 267 (e.g., via outlet 273 (see, FIG. 17), etc.). The blower 298 is configured to generate/provide an air flow within the conduit 290 (via air conveyor 320) that operates to direct (e.g., draw, push, carry, etc.) the pollen from the outlet 273 of the collector 267 to the distributor 294, in substantially the same manner described above for the blower 98, conduit 90, and air conveyor 120 (and with the description of the air conveyor 120 above applying to the air conveyor 320).

The collector 267 generally includes an inlet 271, the separation chamber 272, and the outlet 273. The inlet 271 is coupled to a sidewall 360 of the separation chamber 272, and is generally in fluid communication with the separation chamber 272. In connection therewith, the inlet 271 is generally aligned with, or generally tangent with, the sidewall 360 of the separation chamber 272. Similarly, the outlet 273 is coupled to the sidewall 360 of the separation chamber 272, at a location generally opposite the inlet 271 (e.g., at a location on the sidewall 360 of the separation chamber 272 that is at least about 180 degrees around the sidewall 360 of the separation chamber 272 from the inlet 271, etc.), and is also in fluid communication with the separation chamber 272. In addition, in the illustrated embodiment, the inlet 271 includes a screen 275 (broadly, a filter, etc.) configured to screen out (e.g., inhibit, etc.) leaves and other large debris (e.g., as may be dislodged from the plants and carried in the air flow with the pollen, etc.) from entering the separation chamber 272.

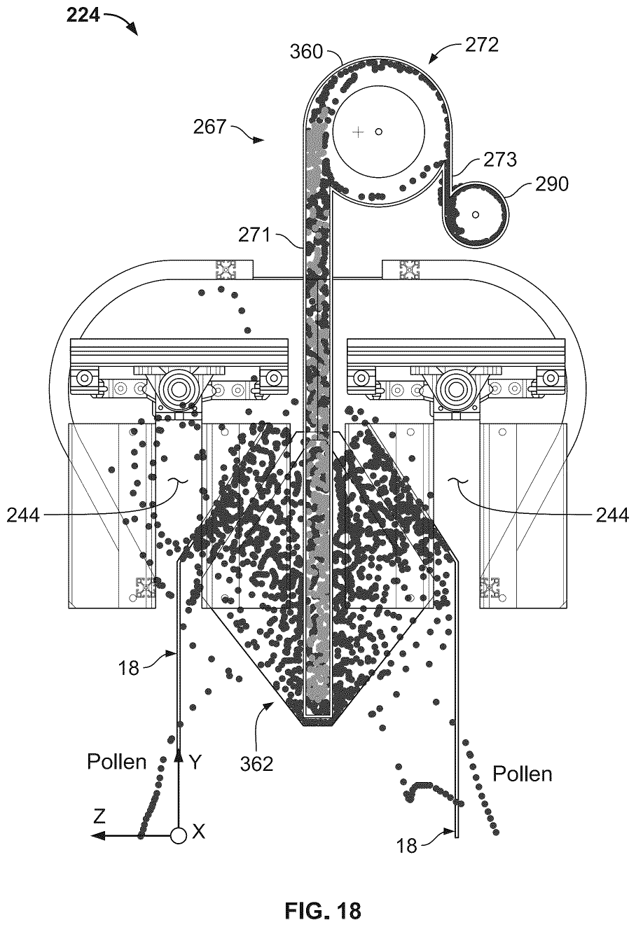

As such, in operation, the air system 264 is configured to generate negative pressure (e.g., a vacuum, etc.) within the separation chamber 272 (and, in doing so, also within the inlet 271, etc.). In particular, the inlet 271 is configured to draw the pollen removed from the pollen bearing plants, at the channels 244 of the collection unit 224, into and through a funnel unit 362 (FIG. 18) in communication with each of the channels 244. In turn, the pollen moves through the inlet 271 and into the separation chamber 272, where the pollen is directed, deflected, etc. (e.g., moves, travels, flows, rides, etc.) along an inner surface of the sidewall 360 (via the air flow generated by the air system 264 (and blower 268)). In connection therewith, the air flow generated by the air system 264 carries the pollen circumferentially around the separation chamber 272 (along the inner surface of the sidewall 360 generally based on the shape of the inner surface of the sidewall 360) (see, FIG. 17). Then, as the pollen reaches the outlet 273, it is directed by the sidewall 360 of the separation chamber 272 into the conduit 290 (e.g., a trunk line portion of the conduit 290 adjacent the separation chamber 272, etc.) of the air system 292. As such, in this embodiment, a generally constant air flow is provided within the separation chamber 272 and inertia forces the pollen to ride along the inner surface of the sidewall 360 of the separation chamber 272, which in turn directs (or guides) the pollen (or puts the pollen on a trajectory) generally toward (or into) the conduit 290 of the air system 292 (via the outlet 273).

Air flow generated by the air system 292, then, generally cushions the pollen as it is received in the conduit 290 (e.g., to inhibit damage to the pollen, etc.) and directs the pollen along the conduit 290 to the distributor 294 in substantially the same manner as described above with regard to the air system 292. In particular, the air system 292 is configured to generate a negative pressure (e.g., a vacuum, etc.) within the conduit 290 that is relatively higher than the negative pressure in the separation chamber 272 (as generated by the air system 264). In this way, the inertia forces acting on the pollen around the sidewall 360 of the separation chamber 272 and the air flow created by the air system 292 within the conduit 290 generally operate together to separate the pollen from the air flow generated by the air system 264 (within the separation chamber 272). The pollen is then directed, by the distributor 294, to the manifold 100 and applicator units 28 in substantially the same manner as described above (with regard to the distributor 94, etc.). In this way, in this embodiment, the pollen generally continuously moves from the channels 244 of the collection unit 224 to the distributor 294 (generally without settling, etc.).

In addition in this embodiment, the generally round and elongate configuration of the separating chamber 272 of the collector 267, in combination with the air flows generated by the air systems 264, 292, facilitates a cyclonic action (e.g., a generally circular air flow, etc.) within the separating chamber 272 such that the pollen generally moves circumferentially around the sidewall 360 of the separating chamber 272 (as described above) (e.g., the pollen is not drawn longitudinally through the separating chamber to the blower 268, etc.). Further, the air system 264 is configured to collect/draw the pollen from the channels 244 of the collection unit 224, and transport the pollen through the separating chamber 272, at a relatively high volumetric flow, while the air system 292 is configured to convey the pollen through the conduit 290 to the distributor 294 at a relatively low volumetric flow (e.g., such that a ratio of air flow generated by the air system 264 to air flow generated by air system 292 is about 10:1, etc.). In this way, the relatively larger volumetric flow generated by the air system 264 enables the pollen removed by the agitators 254 (and agitators 54) to be drawn from the channel 244 (and channel 44) of the collection unit 224 (and collection unit 24) to the collector 267 (and hopper 66). Then, the reduced air flow, at the air system 292, generally allows for centralized collection of the pollen at the distributor 294 (and distributor 94) and uniform distribution of the pollen to the manifold 100 and applicator units 28.

In some embodiments, the pollen received at the distributor 294 (and distributor 94) may be directed from the distributor 294 (and distributor 94) to one or more desired storage containers for treatment, collection, etc. (instead of being directed to the applicator units 28 and/or delivered to pollen-receiving plants, etc.). In this way, the pollination assembly 10 may operate as a pollen treatment and/or collection assembly. In connection therewith, the pollen may be analyzed as part of the collection, for example, via one or more flow rate sensors (e.g., to measure collection rates of pollen in real time, etc.), moisture sensors (e.g., one or more NIR, NMR, or capacitive pollen moisture measurement devices, etc.) (e.g., to measure moisture of the pollen in real time as the pollen is collected, etc.), etc. In addition, one or more additives, treatments, etc. may be added to the pollen as it is being collected, for example, to prepare the pollen for subsequent storage, use, etc.

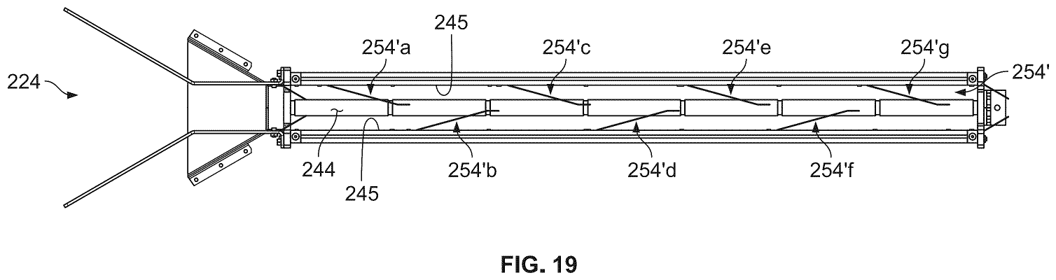

FIGS. 19-20 illustrate an example embodiment of an agitator assembly 254′ (broadly, an agitator) that may be used in the collection unit 224. In connection therewith, one such agitator assembly 254′ is disposed within each of the channels 244 of the collection unit 224 illustrated in FIG. 14 (only one agitator assembly 254′ and one channel 244 are shown in FIG. 19). In this position, the agitator assembly 254′ is configured to agitate tassels of plants as the plants enter the channel 244 of the collection unit 224, to remove pollen from the plants.

In this example embodiment, the agitator assembly 254′ includes multiple individual agitators 254′a-g each having multiple fingers (or tines) 255′ (broadly, protrusions) coupled to a sidewall 245 of the collection unit 224 (via base 257′) (e.g., defining a rake structure, etc.). In this arrangement, as plants are received into the channel 244 of the collection unit 224, and move through the channel 244, the fingers 255′ engage (e.g., operatively contact, etc.) the plants (e.g., tassels of the plants, stalks of the plants, etc.) to facilitate release of pollen therefrom. The air system 264, then, is configured to draw pollen removed from the plants (by the agitators 254′ within the channels 244) into the collector 267, for example, as generally described above.

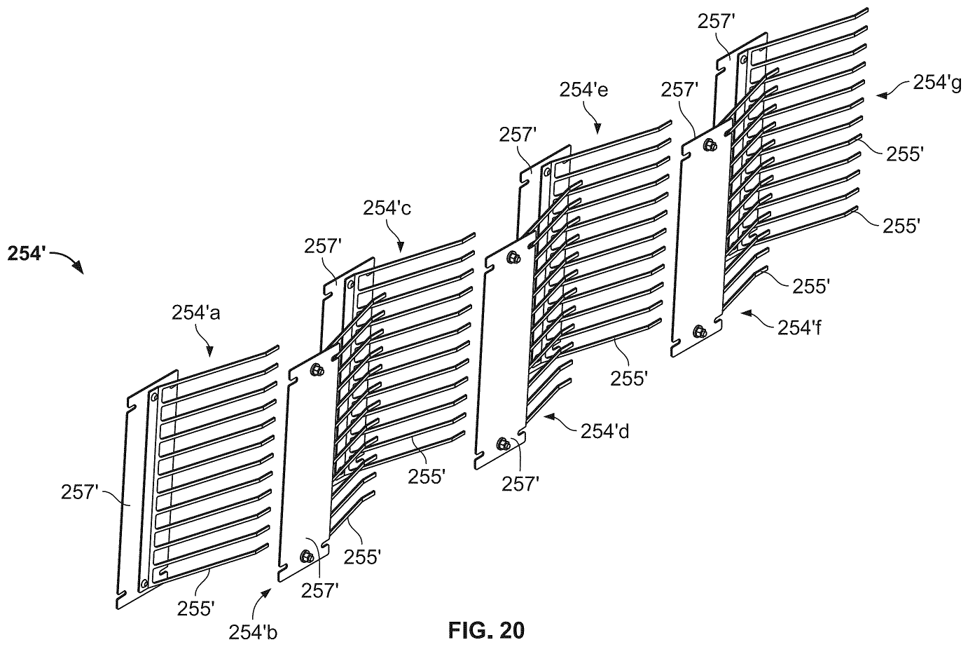

FIG. 21 illustrates an example embodiment of an assembly 410 of the present disclosure suitable for use in collecting pollen from pollen-bearing plants 18 in multiple rows and then storing the pollen for subsequent use (e.g., for use in application/delivery to pollen-receiving plants in a location or field different from the pollen-bearing plants from which the pollen was collected, etc.). The assembly 410 includes multiple collection units 424 coupled to a carriage 422 of a tractor 414. The collection units 424 are each configured to remove pollen from the pollen-bearing plants 18 and then direct the pollen (via corresponding distribution units (e.g., one or more of distribution units 26, 226, etc.), etc.) to a central storage unit 470. In doing so, the collection units 424 are configured to operate in substantially the same manner as described above (with regard to the collection units 24, 224) to dislodge (or remove) pollen from the pollen-bearing plants (e.g., via one or more agitators 54, 254 as generally described above, etc.) and collect the pollen for transport to the storage unit 470.

In particular in this embodiment, each of the collection units 424 is coupled to the storage unit 470 via a corresponding conduit 472. An air system (not shown) may then be provided to direct the pollen through the conduits 472 to the storage unit (e.g., the air system 92, the air system 292, etc. described above; etc.). In doing so, one or more distributors (e.g., distributor 94, distributor 294, etc.) may be used to help control velocity of the pollen as it is delivered to the storage unit 470. The collected pollen may then be analyzed as part of the collection (e.g., in real time while the pollen is being collected, etc.). For example, one or more sensors 474 may be used to measure one or more characteristics of the pollen (e.g., one or more flow rate sensors (e.g., to measure a rate of collection of pollen in real time, etc.), moisture sensors to measure moisture levels of the pollen (e.g., one or more NIR, NMR, or capacitive pollen moisture measurement devices, etc.), sensors to measure an amount/height of pollen in the storage unit 470, etc.). In one example, the sensors 474 may include one or more NIR sensors configured to measure viability of the collected pollen. In example embodiments, the storage unit 470 may be temperature controlled, and/or may be coupled to the tractor via a quick-release connection to facilitate and removal and/or replacement.

In addition, one or more additives may be added to the pollen, from onboard storage 476 (e.g., via conduit 478, etc.), as the pollen is being collected (e.g., in the storage unit 470, etc.), for example, to prepare the pollen for storage, use, etc. In the illustrated embodiment, a sensor 480 is provided to meter desired amounts of the additive (e.g., relative to a flow rate of pollen being received into the storage unit 470, etc.). Alternatively, the desired amounts of the additive may be controlled by a capturing the pollen temporarily in a feeder unit/chamber (e.g., a staging chamber, etc.) that can dose a set rate of pollen collected in the feeder unit/chamber, regardless of collection rate of the pollen, relative to another feeder that is dosing additive at a know rate. Further, in various examples, the desired amounts of the additive to be added to the collected pollen may be based on the amount of pollen collected. Here, the collected pollen may then be dosed, later, as needed with additive.

In some example embodiments, an additional collector (such as collector 267) (e.g., a hopper-less collector, etc.) may be included in the assembly 410 adjacent the storage unit 470 (e.g., in line between the conduits 472 and the storage unit 470, etc.). In such embodiments, the additional collector may be configured to receive the collected pollen from the collection units 424 and separate the pollen from the air flow, so that the separated pollen is then received in the storage unit 470 (in a similar operation to that described for the collector 267 in which the pollen is removed from the air flow within the separation chamber 272 and directed (via inertia, etc.) into the conduit 290).

In some example embodiments, the collection hopper (e.g., collection hopper 66, etc.) of the pollination assembly (e.g., assembly 10, etc.) may be used as a storage unit. In such example embodiments, air locks (e.g., air locks 97, etc.) provided at outlet ports of the collection hopper may be closed to selectively store collected pollen in the collection hopper. As such, when the air locks are closed, pollen is collected in the collection hopper. And, when the air locks are open, the collected pollen is directed to the distributor (e.g., in the manner described above via the conduit 90, etc.). In some examples, the collection hopper may include augers disposed at the air locks, where the augers are in communication with the distributor (e.g., distributor 94, etc.). In such examples, when the air locks are open, the pollen is reintroduced into the distributor via the augers.

In some example embodiments, the storage unit 470 on the tractor 614 may be in communication with each of the collection units 424 via additional conduits (not shown), whereby the collected pollen may be circulated (or recirculated) from the storage unit 470 back to distributors of the collection units 424 (e.g., for subsequent mixing, for subsequent distribution to pollen-receiving plants, etc.). In connection therewith, the distributor (e.g., distributor 94, distributor 294, etc.) of each of the collection units 424 may include a receiving assembly located at an upper portion of the distributor (e.g., at the manifold thereof, etc.) configured to receive the pollen from the storage unit 470 and direct the pollen back into the distributor. The mixed/reintroduced pollen may then be directed from the distributor to the manifold, and to the applicator units for delivery to pollen-receiving plants.

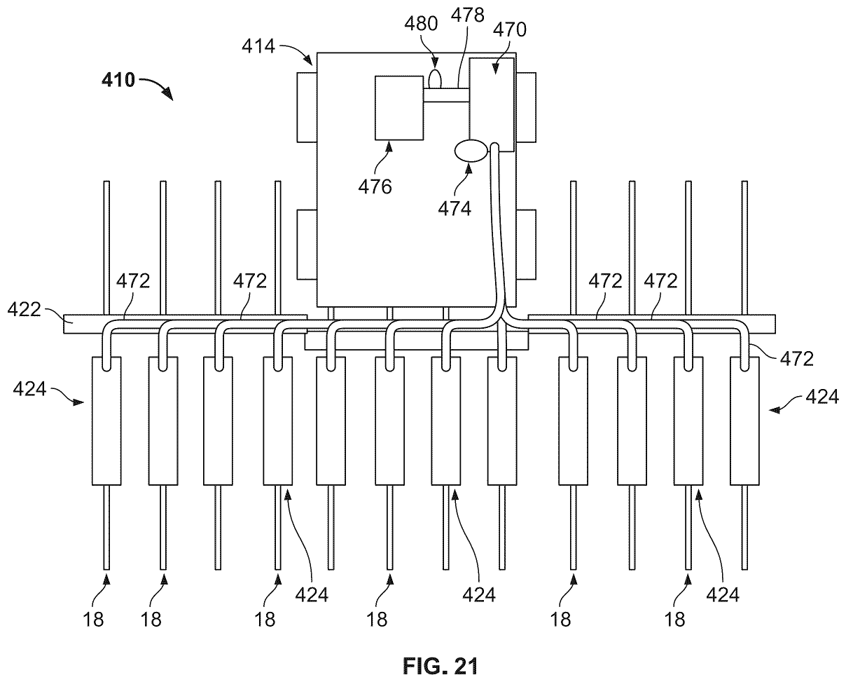

As shown in FIG. 22, in some example embodiments, storage unit 470 may be located on (or may be associated with) a distributor 494 (e.g., of one of the collection units 424, of each of the collection units 424, or separate therefrom, etc.) of the assembly 410. It should be appreciated that the distributor 494 is substantially the same as the distributor 94 and the distributor 294 described above, whereby the descriptions of the distributors 94, 294 provided above should be understood to also apply to the distributor 494 (e.g., such that the distributor 494 may be used in place of the distributors 94, 294, as desired; etc.).

As shown, the storage unit 470 is coupled to a manifold 500 positioned on top of the distributor 494. The manifold 500 is substantially the same as the manifold 100 described above, whereby the description of the manifold 100 provided above should be understood to also apply to the manifold 500. A tube 477 (or conduit or pathway, etc.) of the storage unit 470 is coupled to an upper opening 501 of the manifold 500. As such, pollen to be transferred from the storage unit 470 may be directed from the storage unit 470 into the manifold 500, where it falls into (or enters) the distributor 494 (through the opening 501). In the illustrated embodiment, the storage unit 470 includes a dual twin screw feeder 479 configured to direct the pollen from storage unit 470 to the tube 477. That said, it should be appreciated that other devices may be used to transport/direct the pollen to the tube 477 in other embodiment (e.g., any device capable of moving a powder, etc.). The pollen received in the distributor may then be transferred to the manifold and delivered to the applicator units as previously described (e.g., whereby the stored pollen from the storage unit 470 is delivered to the pollen-receiving plants for pollination, etc.). The storage unit 470 in this embodiment is generally air tight so that air flow is not entering the pollen drop tube 477 (e.g., so that pressure in the storage unit 470 is higher than presser in the distributor 494, etc.).

In some example embodiments, the assembly 410 of the present disclosure may be used for collecting insects from within a field (e.g., instead of pollen, etc.). In connection therewith, the collection units may be configured to capture insects from plants (and from adjacent the plants) and then direct the insects (via corresponding distribution units (e.g., one or more of distribution units 26, 226, etc.), etc.) to the central storage unit 470. In doing so, the collection units 424 may be configured to operate in substantially the same manner as described above (with regard to the collection units 24, 224) to dislodge (or remove) insects from the plants (e.g., via one or more agitators 54, 254 as generally described above, without use of the agitators and via negative pressure, etc.) and collect the insects for transport to the storage unit 470.

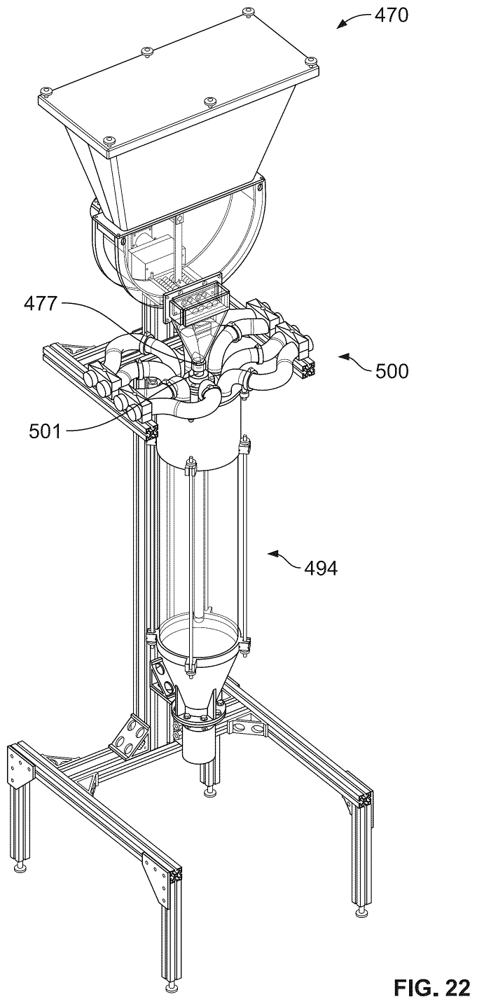

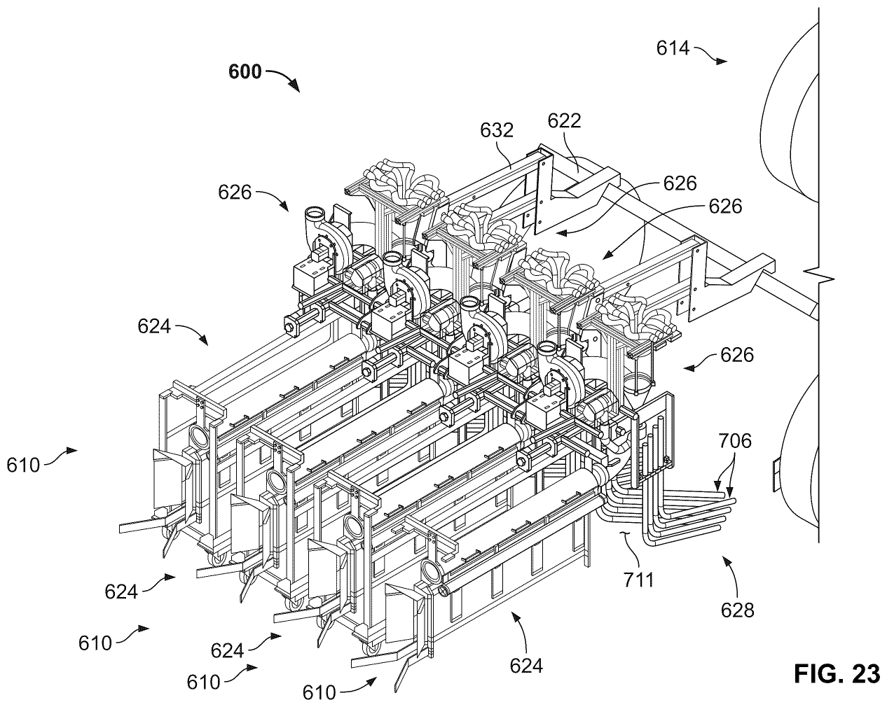

FIGS. 23-24 illustrate another example embodiment of a pollination system 600 of the present disclosure. The pollination system 600 includes multiple pollination assemblies 610, each coupled to a carriage 622 of a tractor 614 (via frame structure 630). In connection therewith, in this embodiment, each of the pollination assemblies 610 is configured to collect pollen from plants (e.g., plants in rows aligned with each of the pollination assemblies 610, etc.), and then direct, move, deliver, convey, transfer, etc. the pollen back to the same plants (in the same rows) from which it was collected (e.g., for effecting self pollination, etc. of the plants as part of an inbred pollination program; etc.). While only four pollination assemblies 610 are shown in FIGS. 23-24, it should be appreciated that the pollination system 600 may include more than four assemblies 610 (or fewer than four assemblies 610) in other example embodiments.

That said, the pollination assemblies 610 of the system 600 are each substantially the same as the pollination assembly 10 (and/or the pollination assembly 410) described above. For instance, each pollination assembly 610 generally includes a collection unit 624, a distribution unit 626, and an applicator unit 628. And, the descriptions above of the collection units 24, 224, 424, the distribution units 26, 226, and the applicator units 28 (and there corresponding parts and operations) also apply to the collection unit 624, the distribution unit 626, and the applicator unit 628 of each assembly 610 (with various differences, modifications, etc. described below and illustrated in FIGS. 23-24).

In this embodiment, each pollination assembly 610 includes a single applicator unit 628 having multiple applicators 706. In particular in the illustrated embodiment, each applicator unit 628 includes eight applicators 706 arranged in four pairs of two applicators 706. In connection therewith, each pair of applicators 706 is generally aligned, and spaced apart, in a vertical direction with the other pairs of applicators 706 (e.g., such that the applicators 706 of one pair are generally vertically aligned with corresponding applicators 706 of another pair, etc.). To this point, the pairs of applicators 706 are each positioned at various different heights to help facilitate (or to help ensure) that pollen distributed from the applicators 706 will be directed at receptive female silks of the plants passing through the pollination assemblies 610 (e.g., the different vertical spacings of the pairs of applicators 706 help account for different heights of plants, different positions of silks on the plants, etc.).

As best shown in FIG. 24, each applicator 706 of each applicator unit 628 includes a tube 708 (e.g., a drop tube, etc.) and a nozzle 710 coupled to an end portion of the tube 708. In addition, the tubes 708 of the applicators 706, of each pair of applicators 706, are arranged relative to each other so as to define a guide for directing, funneling, channeling, etc. the plants (in the given row of plants aligned with the corresponding pollination assembly 610) generally between the applicators 706 of each of the pairs of applicators 706 (e.g., into a pollination zone or window 711 defined between the applicators 706 of each pair, etc.). For example, forward portions of the tubes 708 of each pair of applicators 706 are generally spaced apart to accommodate, facilitate, guide, etc. movement of the plants therebetween. The tubes 708 then angle generally inward (or toward each other for each pair of applicators 706) so that the nozzles 710 of each pair of the applicators 706 are positioned adjacent each other. Further, the nozzles 710 of each pair of applicators 706 are generally directed inwardly toward each other (and at the plants passing between the nozzles 710). In this arrangement, then, the nozzles 710 are configured to provide pollen generally directly at the plants (in a targeted manner, as a directed discharge, etc.) as the plants pass through the corresponding applicator unit 628 (and between the nozzles 710) (e.g., generally targeted at the silks of the plants, etc.).

In each of the pollination assemblies, the applicator unit 628 is coupled to a manifold 700 of the corresponding distribution unit 626. In connection therewith, the manifold 700 includes multiple ports 702, each coupled to one of the applicators 706 of the corresponding applicator unit 628 (via a conduit, etc.). In the illustrated embodiment, the manifold 700 includes eight ports 702, each associated with a corresponding one of the applicators 706 of the applicator unit 628. In addition in this embodiment, each of the ports 702 is associated with (or includes, etc.) a valve 701 configured to selectively block pollen from passing through the port 702 or allow pollen to pass through the port. As such, in operation, the valves 701 associated with a desired applicator 706 or a desired pair of the applicators 706 (or with multiple applicators 706, or with multiple pairs or all pairs of the applicators 706) may be opened, adjusted, etc., as desired, to discharge pollen through the selected pair(s) of applicators 706 (e.g., manually, automatically, etc. via electronic, hydraulic, or other similar means; etc.). In turn, air system 692 of the given assembly 610 may then be configured to transport the pollen delivered to the manifold 700 (from distributor 694) to the selected pair(s) of the applicators 706. In this way, the pollen is discharged from the select applicator(s) 706 at a desired height relative to the plants passing through the pollination assembly 610 and at a desired rate to facilitate targeted delivery of the pollen to the silks of the plants, for example.

In connection with the above, the collection unit 624 of each pollination assembly 610 includes a sensor 638 (e.g., a camera, etc.) positioned generally forward of the collection unit 624 (and forward of the applicator unit 628). The sensor 638 is configured to sense, track, identify, etc. heights of the plants (e.g., including locations of the tassels and/or silks of the plants, etc.), for example, as the tractor 614 moves the pollination assembly 610 through a field and/or as the plants approach and/or are received by the collection unit 624.

In doing so, the sensor 638 may communicate the height of the plants with the collection unit 624, the tractor 614, the applicator unit 628, etc. (e.g., with a computing device thereof, etc.) whereby (in response) the collection unit 624, tractor 614, the applicator unit 628, etc. is configured to adjust (e.g., automatically, etc.) one or more components thereof into position to collect and/or distribute pollen from/to the plants. For instance, in one example, the sensor 638 may communicate the height of the plants and/or positions of silks on the plants to the applicator unit 628 (e.g., to a computing device in communication with the applicator unit 628, etc.). In response, the applicator unit 628 may actuate one or more of the valves of the manifold 700 (of the distribution unit 626) to direct pollen to ones of the applicators 706 located at heights corresponding to the locations of the silks on the plants (e.g., to help ensure that the pollen is directed at the receptive female silks of the plants, etc.).

Further, in some examples, in response to the determination(s) by the sensor 638 (e.g., with regard to locations of the silks of the plants, etc.), the applicator unit 628 may be configured to adjust (e.g., automatically, etc.) a height of the applicators 706 to position the applicators 706 at a desired height to direct pollen at the silks of the plants as they pass through the collection applicator unit 628. For instance, the applicator unit 628 may actuate a portion of a frame supporting the applicators 706 to adjust a height of the applicators 706 (e.g., automatically, etc.) relative to the ground as desired, for example, based on input, communication, etc. from/by the sensor 638, etc. In this manner, the applicators 706 may be particularly positioned to generally align with the silks of the plants, at certain heights, to deliver pollen directly thereto.

That said, example embodiments of the pollination assemblies herein (e.g., assembly 10, assembly 410, etc.) may be used in connection with (or as part of) a seed production program. In doing so, the assemblies herein may facilitate improvements in yield (e.g., though additional fill of seeds, etc.), improvements in reliability of pollination occurrence, expansion of planting patterns, reduction in undesirable seeds (e.g., large rounds in corn seed production, etc.), improvements in seed vigor (e.g., as compared to seeds produced in non-distributed fields, etc.), improvements in seed purity (e.g., as compared to seeds produced in non-distributed fields, etc.), reductions in required land for seed production, reductions in total bag weight with higher proportions of seeds having desired sizes/shapes (e.g., flat corn seeds, etc.), improvements in pollen flow within the crop canopy, etc.

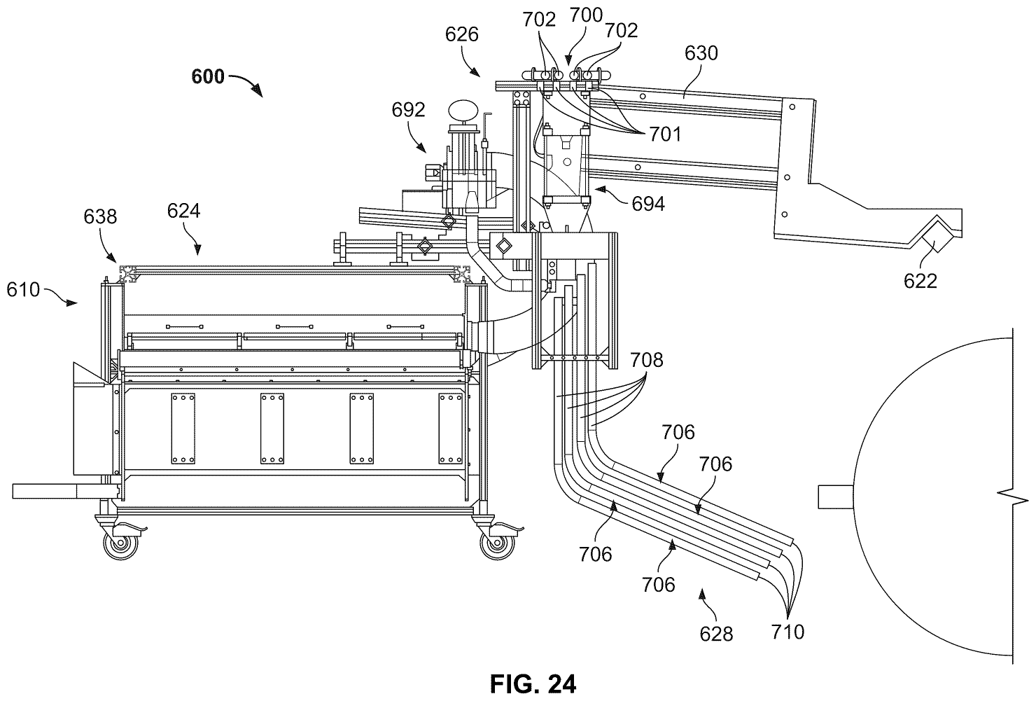

In connection therewith, in some instances, the pollination assemblies herein may be used to facilitate particular seed profiles for use in the seed production program. In particular, the pollination assemblies may be used to shift seed production to seeds having sizes and/or shapes that meet or satisfy one or more desired size and/or shape profile (e.g., to seeds having certain sizes and/or shapes that may be desired and/or that may have higher demand, etc.), over other seeds (e.g., to flat corn seeds over large round corn seeds, etc.). In one particular example, as shown in FIG. 25, the pollination assemblies herein may be used to facilitate seed profiles having a larger number of flat seeds versus round seeds. In this example, two experiments were implemented in which pollen-receiving plants were pollinated using the pollination assemblies herein (“2 App”) (in 4:1 fields), and a control experiment was implemented in which pollen-receiving plants were pollinated without using the pollination assemblies herein (e.g., the plants were pollinated in a conventional manner, etc.) (in 4:1 fields). As shown, the mean percent of flat seeds was increased in the experiments using the pollination assemblies herein.

For example, flat corn seeds may be desirable over large round corn seeds (e.g., for planting, processing, etc.). The shape of the flat corn seeds may allow for better placement in soil, reducing likelihood of seed rotation during planting and helping ensure that the seed remains in optimal position for moisture absorption and nutrient uptake (e.g., which may facilitate improved germination ability and root development, etc.). In addition, the flat shape of such corn seeds may allow for easier handling and distribution by planting equipment (e.g., leading to more consistent planting depth and spacing, healthier plants, higher yields, etc.). In contrast, the shape of round corn seeds may limit ability of the seeds to establish effective contact with the soil, and may adversely affect moisture absorption by the seed (e.g., resulting in reduced germination ability, etc.). And, the larger size of the large round corn seeds may result in increase energy requirements for the seeds to break dormancy and initiate the germination process. Further, large round corn seeds may pose certain seed quality issues (as compared to flat corn seeds), such as, for example, diminished emergence, lower or reduced vigor, higher percentages of internal cracks, higher residual abscisic acid content in fresh harvest (e.g., which may cause secondary dormancy and lead to lower vigor scores in bulk samples, etc.). Moreover, large round seeds may be difficult to handle, without being damaged, during conditioning processes (e.g., due to their position on the cob and exposed embryo face which potentially affects seed quality, etc.), etc.

With that in mind, the assemblies herein may enable redistribution of available pollen in a field, creating greater grain fill, and therefore, an increased quantity and quality of total seeds and total flat seeds being produced from a given field. In particular, through use of the assemblies herein, additional ovules may be pollenated with desired pollen (e.g., pollination may be more complete through use of the assemblies herein as compared to prior art methods of pollination, etc.), such that the occurrence of available silks (or unpollinated silks) capable of receiving pollen from outside contaminant sources is reduced (whereby overall seed purity in the given fields may be improved). As such, the total percentage of flat seeds produced in an average field may be increased, as well as the total seed produced overall.

In connection with the above, implementation of the automated pollen-distribution assemblies herein may decrease the average maximum dimension of any residual round kernels that may still develop after treatment, thereby improving overall seed size uniformity. As an example, field data collected during the 2024 growing season (e.g., as collected using technology described in US 2020/0086353 (which is incorporated herein by reference) demonstrated that a dual-pass application of the assembly lowered the least-squares mean longest-axis measurement of round seeds from approximately 7.44 mm (millimeters) in untreated controls to approximately 7.33 mm, by measuring across the face of the seed, a statistically significant reduction (t≈−2.58; p≈0.012). Therefore, high-efficiency transfer of viable pollen to previously under-pollinated silks promotes more complete fertilization throughout the ear, especially in apical zones typically associated with large rounds. As a result, a greater number of developing kernels compete for limited cob volume, naturally limiting the radial expansion of individual kernels and producing smaller, more manageable round seed fractions. By shrinking the residual round class in this manner, the assemblies (and the pollination effected thereby) reduce conditioning losses, lower mechanical breakage risk during processing, and yield a higher proportion of commercially desirable seed grades without requiring genetic modification or additional chemical inputs.

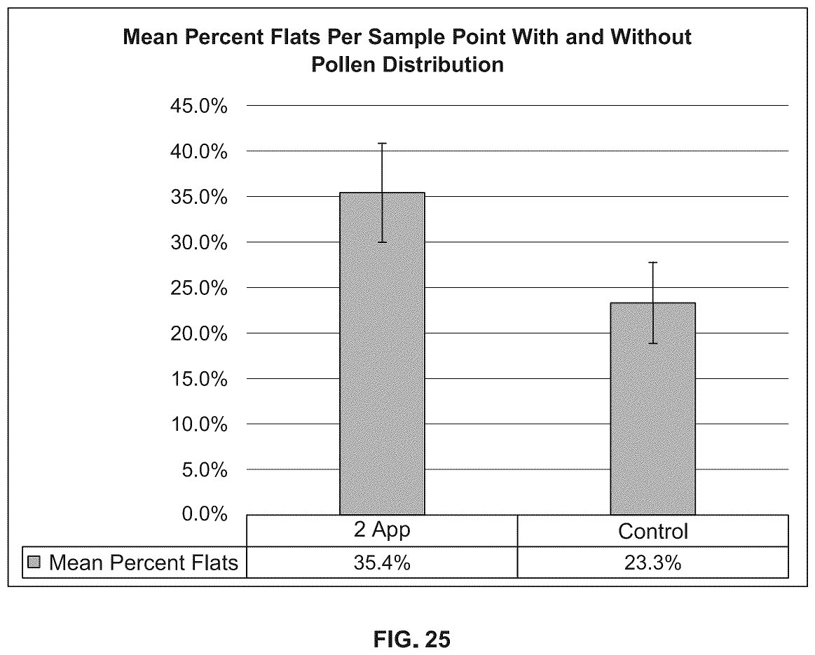

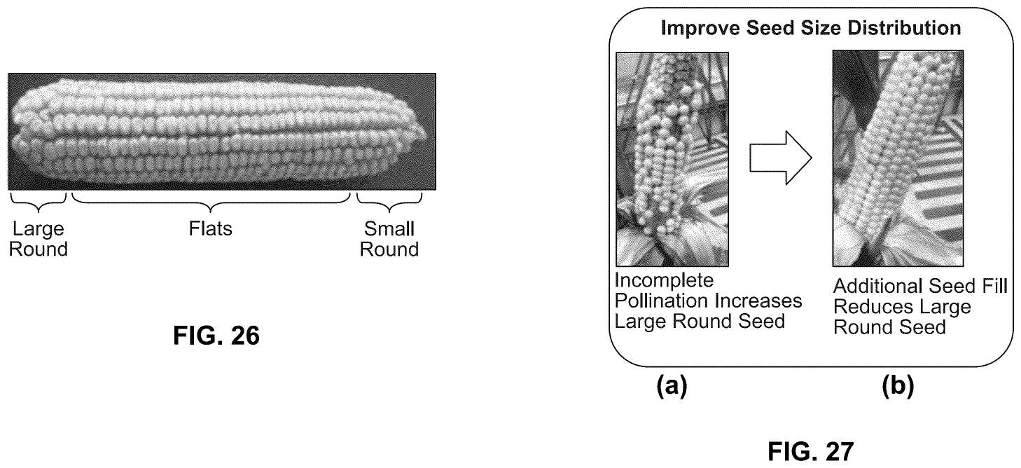

To this point, the pollination assemblies herein may improve yield through additional grain fill created when pollen is moved from the tassels to the silks where they can complete the pollination of unpollinated silks. The assemblies create pollen movement that enables enhanced and/or expanded planting pattern panels, such as: 4:1, 4:2, 6:1 and 9:1, etc. (female plants to male plants). In doing so, the improved grain fill facilitated by the assemblies results in a reduction of large round seeds in a field, since there will be fewer unpollinated ovules (which unpollinated ovules typically allow space for the large seeds to grow on the cob). As such, seeds that would have grown in as large rounds, when additional space on the cob is available, will instead grow in as flat seed since the seed is limited in the available growing space on the cob (following more complete pollination via the assemblies herein). This additional grain fill may also create an improved reliability in seed returns, which can result in the need for fewer acres planted to achieve a desired seed return. In connection therewith, FIG. 26 illustrates example distribution of different seeds on a cob of corning, including large round seeds, flat seeds, and small round seeds. And, FIG. 27 illustrate example corn seed production on a corn cob resulting from pollen redistribution achieved by the assemblies herein (having additional seed fill) (at (b) in FIG. 27), as compared to corn seeds produced on a cob resulting from conventional, prior art pollination (having incomplete pollination and increased production of large round seeds) (at (a) in FIG. 27).

In addition, in some instances, the pollination assemblies herein may be used to facilitate particular (or desired) field operations. For example, the pollination assemblies are operable to improve reliability in available seed and increase seed quality, thereby reducing the amount of land required to obtain target seed amounts (e.g., more seeds may be produced per acre of land, etc.). Further, with regard to corn, by reducing the number of large round seeds per ear, which generally have an increased total weight and volume as compared to flat seeds, overall seed bag weights may be reduced (while still providing the same number of seeds) (e.g., resulting in less mass to move, store, cool, etc. for seed production entities; etc.).

To this point, in corn seed production, 4:2 (female plants to male plants) plantings are often used in hybrid programs, which have a difficult time making seed. However, many seed production fields are grown as 4:1 plantings, making the 4:2 fields a complicating factor for field operations. Planting these difficult hybrids as 4:1 (consistent more conventional field operations), and then using the pollination assemblies herein, seeds having the desired sizes and/or shapes may still be generated for the hybrid programs. For example, a ten (10) acre field that would have been planted at 4:2, may instead be planted at 4:1 and with utilization of the pollination assemblies herein, may obtain about 10-20% more capacity to grow female plants, depending on exact configurations of the planting patterns and/or field shapes.

Further, in some instances, the pollination assemblies herein may also facilitate particular (or desired) facility operations, by mitigating mechanical damage risk to handling/processing of seeds. In particular, in corn production facility operations, large round seeds may be more susceptible to mechanical damage due to application of uneven pressure to the seeds, which may case cracking or splitting of the seed coat and in turn compromising integrity and/or viability of the seed. By promoting certain seed sizes and/or shapes (e.g., certain seed profiles, etc.), that include increased numbers of flat seeds, such risk of damage to seeds may be reduced.

Moreover, in some instances, the pollination assemblies herein may facilitate specific seed production workflows by producing more seeds in a foundation production stage, thereby enabling crosses coming from short males (short corn lines), or improving pollen flow in Cytoplasmic Male Sterility and Roundup Hybridization System (CMS/RHS) fields (e.g., fields in which there is not detasseling since the female plants are male sterile from either CMS or from being sprayed by ROUNDUP at a prior reproductive stage, etc.). where there is additional plant canopy that the pollen would have to navigate through compared to detasseled fields. Generally, optimized pollen diffusion occurs from taller male plants to a set of shorter female plants. Establishing a corn hybrid seed production program where shorter male parents (compared to female parents) are used to produce the hybrid seed is a challenging task. Use of the pollination assemblies herein allows for efficient pollination even where the female plants are taller than the male plants, by making independent height adjustments of the collection and applicator units of the assemblies (as generally described herein).

Taking into account the above, example embodiments of the present disclosure relate to methods for improving and/or increasing corn hybrid seed production in a field. In some examples, such a method includes: (a) planting male and female parent corn plants in a field, wherein the female parent corn plants are characterized as being taller than the male parent corn plants; (b) collecting pollen from the male parent corn plants using one or more of the pollination assemblies herein; (c) distributing the collected pollen to the female parent corn plants; and (d) quantifying (e.g., via yield measurements (e.g., determining a yield of seed produced by the pollinated plants, etc.), based on total weight produced, for instance, as described in US Pat. Publ. No. 2021/0092900 (which is incorporated herein by reference); etc.) hybrid seed production, wherein the hybrid seed production is increased with respect to a control planting (e.g., the exact same planting, but without mechanically distributed pollen applied; etc.). In some examples, the method further includes: (e) observing specific heights of the male and female parent corn plants, and then (f) adjusting collection and/or application portions/units of the pollination assemblies (e.g., heights thereof, etc.) based on the observed heights (e.g., to facilitate pollen collection and distribution, etc.).

In addition to (and/or in connection with) the foregoing, the pollination assemblies herein provide a uniquely advantageous solution for the emerging generation of “short-stature” corn hybrids, whose reduced plant height—and correspondingly lower tassel position—can substantially limit natural pollen dispersal within a conventional canopy. An example of this can be found in Bayer's PRECEON brand smart corn. By mounting the collection and applicator units on independently height-adjustable supports and integrating forward-looking sensors that determine tassel and silk height in real time, the assemblies are capable of lowering the collection unit into precise alignment with the shortened male tassels while simultaneously positioning the corresponding applicator units (e.g., the nozzles thereof, etc.) at the optimal elevation above the silks of adjacent, taller female rows. A first air system then removes viable pollen from the short male tassels without damaging the plants, and a second air system immediately conveys, mixes, and redistributes that pollen across the targeted female silks at controlled velocities that are independent of, and not constrained by, the diminished natural pollen shed associated with short-stature plants. This active redistribution compensates for the inherently shorter pollen flight distance of such hybrids, eliminates reliance on natural wind currents, and results in more complete and uniform fertilization, thereby preserving yield potential, improving seed purity, and enabling producers to exploit the agronomic benefits of short corn—including improved standability and simplified in-season management—without sacrificing pollination efficiency or hybrid performance.

In some examples, a method for improving and/or increasing corn hybrid seed production in a field may include: (a) planting male and female parent corn plants in a field; (b) collecting pollen from the male parent corn plants using the pollen one or more of the pollination assemblies herein; (c) distributing the collected pollen to the female parent corn plants; and (d) quantifying hybrid seed production, wherein the hybrid seed production purity level (e.g., as determine by DNA fingerprinting, wherein single nucleotide polymorphisms (SNPs) which act as unique markers to distinguish between different genetic backgrounds, are profiled; etc.) is increased with respect to a control planting.

Example embodiments of the present disclosure also relate to reducing variations in seed size of hybrid seeds produced in a field. In some examples, such a method includes: (a) planting male and female parent corn plants in a field; (b) collecting pollen from the male parent corn plants using one or more of the pollination assemblies herein; (c) distributing the collected pollen to the female parent corn plants; and (d) determining a size profile of hybrid seeds produced by the pollenated female parent corn plants (e.g., a number of flat seeds produced, a ration of flat to round seeds produced, etc.), wherein the hybrid seeds produced have at least an increase in flat seeds with respect to a control planting. In various examples, the hybrid seeds produced have an increase of about 1-10%, about 10-20%, about 20-30%, or about 30-40%, etc., in flat seeds with respect to the control planting.

In some examples, as generally described above, the pollination assemblies herein may be used for inbred seed production for foundation seed. Foundation seed, within a seed production entity, is the second generation of seed produced from a new plant variety, which is directly derived from seed produced in a breeding program. Standard methods of foundation seed production may include planting inbred seed and allowing for natural self-pollination within the field. Foundation seed purity is important for high quality seed. Purity issues arise when foreign pollen coming into a foundation seed batch field pollenates plants. Seed production operations take various measures to ensure seed purity, including planting at a greater than conventional distance to minimize the probability of foreign pollen entering the field from the environment. The pollination assemblies herein enable high purity seed by reducing the probability of non-target pollen having exposure to receptive silks. To this point, in a field that has utilized the pollination assemblies herein, pollen may be more efficiently applied to and may more completely cover the silks of the female receptive plants, which reduces available receptive silks for foreign pollen.

In connection therewith, example embodiments of the present disclosure relate to methods of producing foundation seed, comprising: (a) planting and growing inbred plants in a field within a line; (b) collecting pollen from the inbred plants using one or more of the pollination assemblies herein; (c) distributing the collected pollen within the line of plants to the silks within the same line using the pollination assemblies herein; and (d) quantifying foundation seed production, wherein foundation seed production purity level is increased with respect to a control planting. In some examples, the hybrid seed produced has an increase of about 1-10%, about 10-20%, about 20-30%, about 30-40%, etc. in flat seeds with respect to a control planting. In some examples, the isolation distances are reduced by about 1-10%, about 10-20%, about 20-30%, about 30-40%, etc.

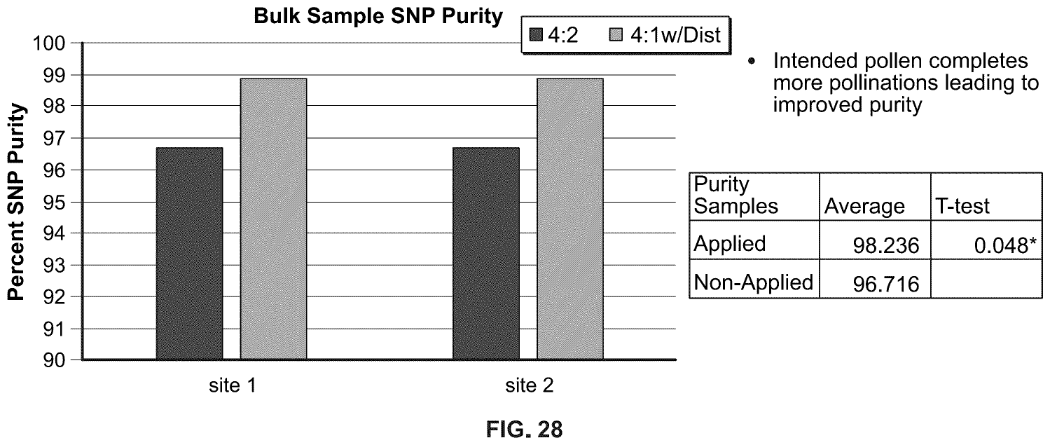

In the above examples, seed purity may be calculated using a single nucleotide polymorphisms (SNP) testing method (e.g., as is generally standard within the seed production industry, etc.). In doing so, a 95% purity threshold may be required for base genetics. FIG. 28 illustrates a graph of seed purity showing a statistically significant increase to the purity of the seed that was created using the pollination assemblies and methods of the present disclosure. In connection therewith, two independent sites where evaluated, which included a standard practice 4:2 (female: male) planting of difficult to produce hybrid seed, and a 4:1 (female: male) of the same lines were planted and then a pollination assembly of the present disclosure was deployed to enhance pollination efficiency. As shown, the pollination assembly improved overall pollination efficiency and when seed purity is measured, there was statistically significant improvement via SNP testing. In addition, as described, the assemblies herein help inhibit silks from being contaminated by foreign pollen (non-target or off target pollen) from the environment.

To this point, a higher SNP purity generally indicates a higher degree of genetic conformity and consistency in the seed, which may translate into better performance and yield in the field. As shown in FIG. 28, the use of the pollination assemblies described herein significantly increased the SNP purity of the seed produced in a field, as compared to a control planting without the use of such a assemblies. For example, in two sites where the pollination assemblies were applied, the average SNP purity of the bulk samples was 98.236%, which was statistically higher than the average SNP purity of the non-applied samples, which was 96.716%. This represents an improvement of about 1.5% in the SNP purity, which may have a substantial impact on the seed quality and value.

In instances where the pollination assemblies described herein are used across an entire seed production organization, the potential benefits may be even more pronounced. For instance, assuming a conservative estimate of 1% improvement in the SNP purity across all the seed production fields, this could result in an increase of about 2% in the overall seed quality for the organization. This could also translate into a significant increase in the market value and competitiveness of the seed products, as well as a reduction in the production costs and risks associated with seed contamination and variability. Therefore, the pollination assemblies disclosed herein provide a valuable tool for enhancing the seed quality and efficiency in the seed production industry.

Example embodiments of the present disclosure also provide for reducing pollen contamination within the pollination assemblies herein. Pollen cross contamination is an issue within seed production that arises when pollen from plants, which are not specifically selected to act as the male donor plant, fertilize the female lines used for seed increase. Various measures may be taken when producing inbred lines to avoid cross contamination, including having established isolation distances between fields and having planting times staggered so the males producing pollen are less likely to pollenate off target females.

To ensure there is not cross contamination within the pollination assemblies herein, the assemblies may be time delayed by at least about one hour to ensure any remaining pollen within the system is unviable. Alternatively, or additionally, physical measures may be taken such as spraying out the assemblies with a cleaner. In one embodiment, heating elements are placed in front of the air distribution systems to heat the air circulated through the pollination assemblies, as well as heating the pollination assemblies themselves, to the point at which pollen is killed or otherwise unviable for germinating the female silks.

In some examples, the pollination assemblies herein may be used in on-seed production fields, where hybrid seed has been planted, to help ensure yield is obtained for the fields.

In some examples, fertile and sterile seed may be produced in a field. In current standard practices, a seed producer may grow 6 rows by 6 rows, where the pollen from fertile plants fertilizes the sterile plants. This is called sterile increase, and also is a selfing of the fertile plants. The pollination assemblies herein may enable for increasing seed set and targets in a smaller land area. For instance, the pollination assemblies herein may be used to achieve sterile increase, where planting patterns may be 4:2, 4:1, and expanded panels 6:1 and 9:1 (as opposed to a 6:6 planting pattern).

In certain seed production fields that utilize sterility technology, such as CMS or RHS, the canopy may be a factor in reducing the amount of pollen shed that reaches the receptive silks. In connection therewith, example embodiments of the present disclosure relate to methods of pollenating male sterile plants within in a field, comprising: (a) planting male sterile female corn plants in a field and planting a non-sterile male line in the field, in a 4:1, 6:1, 9:1 planting pattern; (b) collecting pollen from the male parent plants via one or more of the pollination assemblies herein; (c) distributing the collected pollen to the female corn plants; and (d) quantifying hybrid seed production, wherein hybrid seed production total seed is increased with respect to a control planting.

Still further to the above, example characteristics that may be targeted in connection with use of the pollination assemblies herein may include, as examples, lines of hard to produce pollen, low pollen quantities, heat stressed male lines (e.g., during periods of high heat, pollen is susceptible to death, whereby use of the pollination assemblies herein may allow for remaining viable/living pollen to reach target silks and reduce risk of field loss; etc.), low wind during pollination window.

In addition, within seed production, generally there are a limited number of male lines available because male lines have pollen production and combining ability problems. The pollination assemblies herein provide a tool for transporting available pollen to receptive silks (in the same or different fields, etc.).

In connection therewith, example embodiments of the present disclosure relate to methods of pollenating fields in which (or where) no pollen-producing (e.g., male, etc.) plants have been planted, comprising: (a) planting and growing male inbred plants in a first field and female inbred plants in an alternative (or different) second field; (b) collecting pollen from the male inbred plants in the first field using one or more of the pollination assemblies herein; (c) storing the collected pollen in a storage assembly (e.g., onboard or not onboard the pollination assemblies, etc.); and (d) distributing the stored pollen to the silks of the female inbred plants in the second field using the pollination assemblies herein.

Example embodiments of the present disclosure may further include the following:

Embodiment 1. A pollination assembly for use in transferring pollen between plants, the pollination assembly comprising: a collection unit configured to dislodge pollen from pollen-bearing plants, the collection unit including a first air system configured to direct the dislodged pollen from the pollen-bearing plants to an outlet of the collection unit; at least one applicator unit configured to direct the dislodged pollen received from the pollen-bearing plants at pollen-receiving plants; and a distribution unit disposed adjacent the collection unit, the distribution unit including a second air system configured to direct the dislodged pollen from the outlet of the collection unit to the at least one applicator unit.

Embodiment 2. The pollination assembly of Embodiment 1, wherein the distribution unit includes a distributor disposed between the outlet of the collection unit and the at least one applicator unit, the distributor configured to decelerate the dislodged pollen received, via the second air system, from the outlet of the collection unit.

Embodiment 3. The pollination assembly of Embodiment 1, wherein the collection unit defines at least one channel for receiving the pollen-bearing plants into the collection unit.

Embodiment 4. The pollination assembly of any one of Embodiments 1-3, wherein the collection unit includes at least one agitator configured to engage the pollen-bearing plants to thereby dislodge the pollen from the pollen-bearing plants.

Embodiment 5. The pollination assembly of any one of Embodiments 1-4, wherein the collection unit includes at least one guide configured to facilitate movement of the pollen-bearing plants into the collection unit.

Embodiment 6. The pollination assembly of any one of Embodiments 1-5, wherein the second air system includes an air conveyor configured to generate an air flow to direct the dislodged pollen from the outlet of the collection unit to the at least one applicator unit.

Embodiment 7. The pollination assembly of Embodiment 6, wherein the air conveyor includes a body and an air plenum coupled to the body, and wherein the body and the air plenum define a discharge extending circumferentially around the air conveyor and configured to generate the air flow.

Embodiment 8. The pollination assembly of Embodiment 7, wherein the air plenum is moveable relative to the body to adjust a size of the discharge.

Embodiment 9. The pollination assembly of any one of Embodiments 1-5, wherein the collection unit includes a separation chamber configured to separate the dislodged pollen from an air flow associated with the first air system and direct the separated pollen to the outlet of the collection unit.

Embodiment 10. The pollination assembly of Embodiment 9, wherein the collection unit incudes a closure at the outlet; and wherein the separation chamber is configured to store the dislodged pollen within the separation chamber when the closure is in a closed position.

Embodiment 11. The pollination assembly of Embodiment 9, wherein the separation chamber is configured to separate the dislodged pollen from an air flow associated with the first air system based, at least in part, on inertia of the dislodged pollen.

Embodiment 12. The pollination assembly of any one of Embodiments 1-11, wherein the at least one applicator unit includes at least one nozzle configured to discharge the pollen at the pollen-receiving plants and at least one duct coupled to the nozzle, the at least one duct configured to redirect at least some of the pollen discharged by the nozzle at the pollen-receiving plants.

Embodiment 13. The pollination assembly of Embodiment 1, further comprising at least a third air system arranged in series with the second air system; wherein the at least a third air system is configured to operate in conjunction with the second air system to direct the dislodged pollen from the outlet of the collection unit to the at least one applicator unit.

Embodiment 14. The pollination assembly of Embodiment 13, wherein the distribution unit includes a first distributor and a second distributor; wherein the first distributor is disposed between the outlet of the collection unit and the second distributor, the first distributor configured to decelerate the dislodged pollen received, via the second air system, from the outlet of the collection unit; and wherein the second distributor is disposed between the first distributor and the at least one applicator unit, the second distributor configured to further decelerate the dislodged pollen received, via the at least a third air system, from the first distributor of the distribution unit.

Embodiment 15. The pollination assembly of Embodiment 13, wherein the at least a third air system includes an air conveyor configured to generate an air flow to direct the dislodged pollen from the outlet of the collection unit to the at least one applicator unit.

Embodiment 16. A tractor comprising the pollination assembly of any one of Embodiments 1-15.

Embodiment 17. A pollination system, comprising: a tractor having a carriage; and the pollination assembly of any one of Embodiments 1-15 coupled to the carriage of the tractor.

Embodiment 18. A method for collecting and transferring pollen between plants, the method comprising: receiving at least one pollen-bearing plant into a collection unit of a pollination assembly; dislodging, by at least one agitator, pollen from the at least one pollen-bearing plant within the collection unit; directing, by a first air system, the dislodged pollen from the pollen-bearing plant to an outlet of the collection unit; receiving, by at least one distributor, the dislodged pollen from the outlet of the collection unit; decelerating the dislodged pollen within the at least one distributor; and directing, by at least a second air system, the dislodged pollen to at least one applicator unit for transfer to at least one pollen-receiving plant.

Embodiment 19. The method of Embodiment 18, wherein receiving the at least one pollen-bearing plant into the collection unit includes directing, by at least one guide, the at least one pollen-bearing plant into a channel of the collection unit and then receiving the at least one pollen-bearing plant into the collection unit through the channel.

Embodiment 20. The method of Embodiment 18 or Embodiment 19, wherein dislodging, by the at least one agitator, the pollen from the at least one pollen-bearing plant within the collection unit includes contacting the at least one pollen-bearing plant with the at least one agitator.

Embodiment 21. The method of any one of Embodiments 18-20, further comprising: prior to directing the dislodged pollen to at least one applicator unit for transfer to at least one pollen-receiving plant, directing the dislodged pollen to a storage unit; and then directing the stored pollen from the storage unit to the at least one distributor; mixing the stored pollen with further dislodged pollen received from the outlet of the collection unit; and directing the mixed stored pollen and dislodged pollen to the at least one pollen-receiving plant.

Embodiment 22. The method of any one of Embodiments 18-21, wherein directing, by a first air system, the dislodged pollen from the pollen-bearing plant to the outlet of the collection unit includes generating a first air flow, by the first air system, and transporting the dislodged pollen from the pollen-bearing plant to the outlet of the collection unit via the first air flow; wherein directing, by at least a second air system, the dislodged pollen to the at least one applicator unit includes generating a second air flow, by the at least a second air system, and transporting the dislodged pollen to the at least one applicator unit via the second air flow.

Embodiment 23. The method of Embodiment 21, wherein the second air flow is less than the first air flow.

Embodiment 24. The method of Embodiment 22 or Embodiment 23, further comprising separating, by a separating chamber, the dislodged pollen from the first air flow and directing the separated pollen to the outlet of the collection unit.

Embodiment 25. An assembly for collecting pollen, the assembly comprising: a collection unit configured to dislodge pollen from pollen-bearing plants, the collection unit including a first air system configured to direct the dislodged pollen from the pollen-bearing plants to an outlet of the collection unit; a storage unit; and a distribution unit disposed adjacent the collection unit, the distribution unit including a second air system configured to direct the dislodged pollen from the outlet of the collection unit to the storage unit.

Embodiment 26. The assembly of Embodiment 25, wherein the distribution unit includes a distributor disposed between the outlet of the collection unit and the storage unit, the distributor configured to decelerate the dislodged pollen received, via the second air system, from the outlet of the collection unit.

Embodiment 27. The assembly of Embodiment 26, wherein the collection unit defines at least one channel for receiving the pollen-bearing plants into the collection unit.

Embodiment 28. The assembly of any one of Embodiments 25-27, wherein the collection unit includes at least one agitator configured to engage the pollen-bearing plants to thereby dislodge the pollen from the pollen-bearing plants.

Embodiment 29. The assembly of any one of Embodiments 25-28, wherein the second air system includes an air conveyor configured to generate an air flow to direct the dislodged pollen from the outlet of the collection unit to the at least one applicator unit.

Embodiment 30. The assembly of any one of Embodiments 25-29, wherein the collection unit includes a separation chamber configured to separate the dislodged pollen from an air flow associated with the first air system and direct the separated pollen to the outlet of the collection unit.

Embodiment 31. The assembly of Embodiment 30, wherein the separation chamber is configured to separate the dislodged pollen from an air flow associated with the first air system based, at least in part, on inertia of the dislodged pollen.

Embodiment 32. The assembly of Embodiment 30 or Embodiment 31, wherein the storage unit includes the separation chamber.

Embodiment 33. The assembly of any one of Embodiments 25-32, further comprising at least one sensor configured to measure at least one characteristic of the pollen, in real time, as the pollen is received in the storage unit.

Embodiment 34. The pollination assembly of Embodiment 25, further comprising at least a third air system arranged in series with the second air system; wherein the at least a third air system is configured to operate in conjunction with the second air system to direct the dislodged pollen from the outlet of the collection unit to the storage unit.

Embodiment 35. The pollination assembly of Embodiment 34, wherein the distribution unit includes a first distributor and a second distributor; wherein the first distributor is disposed between the outlet of the collection unit and the second distributor, the first distributor configured to decelerate the dislodged pollen received, via the second air system, from the outlet of the collection unit; and wherein the second distributor is disposed between the first distributor and the storage unit, the second distributor configured to further decelerate the dislodged pollen received, via the at least a third air system, from the first distributor of the distribution unit.

Embodiment 36. The pollination assembly of Embodiment 34, wherein the at least a third air system includes an air conveyor configured to generate an air flow to direct the dislodged pollen from the outlet of the collection unit to the storage unit.

Embodiment 37. A tractor comprising the pollination assembly of any one of Embodiments 25-36.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

Example embodiments have been provided so that this disclosure will be thorough, and will fully convey the scope to those who are skilled in the art. Numerous specific details are set forth such as examples of specific components, assemblies, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, components, and/or groups thereof, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” and the phrase “at least one of” includes any and all combinations of one or more of the associated listed items.

Although the terms first, second, third, etc. may be used herein to describe various elements, components, seeds, members and/or sections, these elements, components, seeds, members and/or sections should not be limited by these terms. These terms may be only used to distinguish one element, component, seed, member or section from another element, component, seed, member or section. Terms such as “first,” “second,” and other numerical terms when used herein do not imply a sequence or order unless clearly indicated by the context. Thus, a first element, component, seed, member or section discussed below could be termed a second element, component, seed, member or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A pollination assembly for use in transferring pollen between plants, the pollination assembly comprising:
a collection unit configured to dislodge pollen from pollen-bearing plants, the collection unit including a first air system configured to direct the dislodged pollen from the pollen-bearing plants to an outlet of the collection unit;
at least one applicator unit configured to direct the dislodged pollen received from the pollen-bearing plants at pollen-receiving plants; and
a distribution unit disposed adjacent the collection unit, the distribution unit including a second air system configured to direct the dislodged pollen from the outlet of the collection unit to the at least one applicator unit.

2. The pollination assembly of claim 1, wherein the distribution unit includes a distributor disposed between the outlet of the collection unit and the at least one applicator unit, the distributor configured to decelerate the dislodged pollen received, via the second air system, from the outlet of the collection unit.

3. The pollination assembly of claim 1, wherein the collection unit includes at least one agitator configured to engage the pollen-bearing plants to thereby dislodge the pollen from the pollen-bearing plants.

4. The pollination assembly of claim 1, wherein the second air system includes an air conveyor configured to generate an air flow to direct the dislodged pollen from the outlet of the collection unit to the at least one applicator unit; and
wherein the air conveyor includes a body and an air plenum coupled to the body, and wherein the body and the air plenum define a discharge extending circumferentially around the air conveyor and configured to generate the air flow.

5. The pollination assembly of claim 4, wherein the air plenum is moveable relative to the body to adjust a size of the discharge.

6. The pollination assembly of claim 1, wherein the collection unit includes a separation chamber configured to separate the dislodged pollen from an air flow associated with the first air system and direct the separated pollen to the outlet of the collection unit.

7. The pollination assembly of claim 6, wherein the collection unit incudes a closure at the outlet; and wherein the separation chamber is configured to store the dislodged pollen within the separation chamber when the closure is in a closed position.

8. The pollination assembly of claim 1, wherein the at least one applicator unit includes at least one nozzle configured to discharge the pollen at the pollen-receiving plants and at least one duct coupled to the nozzle, the at least one duct configured to redirect at least some of the pollen discharged by the nozzle at the pollen-receiving plants.

9. The pollination assembly of claim 1, further comprising at least a third air system arranged in series with the second air system;
wherein the at least a third air system is configured to operate in conjunction with the second air system to direct the dislodged pollen from the outlet of the collection unit to the at least one applicator unit.

10. The pollination assembly of claim 9, wherein the distribution unit includes a first distributor and a second distributor;
wherein the first distributor is disposed between the outlet of the collection unit and the second distributor, the first distributor configured to decelerate the dislodged pollen received, via the second air system, from the outlet of the collection unit; and
wherein the second distributor is disposed between the first distributor and the at least one applicator unit, the second distributor configured to further decelerate the dislodged pollen received, via the at least a third air system, from the first distributor of the distribution unit.

11. A method for collecting and transferring pollen between plants, the method comprising:
receiving at least one pollen-bearing plant into a collection unit of a pollination assembly;
dislodging, by at least one agitator, pollen from the at least one pollen-bearing plant within the collection unit;
directing, by a first air system, the dislodged pollen from the pollen-bearing plant to an outlet of the collection unit;
receiving, by at least one distributor, the dislodged pollen from the outlet of the collection unit;
decelerating the dislodged pollen within the at least one distributor; and
directing, by at least a second air system, the dislodged pollen to at least one applicator unit for transfer to at least one pollen-receiving plant.

12. The method of claim 11, wherein receiving the at least one pollen-bearing plant into the collection unit includes directing, by at least one guide, the at least one pollen-bearing plant into a channel of the collection unit and then receiving the at least one pollen-bearing plant into the collection unit through the channel; and
wherein dislodging, by the at least one agitator, the pollen from the at least one pollen-bearing plant within the collection unit includes contacting the at least one pollen-bearing plant with the at least one agitator.

13. The method of claim 11, further comprising:
prior to directing the dislodged pollen to at least one applicator unit for transfer to at least one pollen-receiving plant, directing the dislodged pollen to a storage unit; and then
directing the stored pollen from the storage unit to the at least one distributor;
mixing the stored pollen with further dislodged pollen received from the outlet of the collection unit; and
directing the mixed stored pollen and dislodged pollen to the at least one pollen-receiving plant.

14. The method of claim 11, wherein directing, by a first air system, the dislodged pollen from the pollen-bearing plant to the outlet of the collection unit includes generating a first air flow, by the first air system, and transporting the dislodged pollen from the pollen-bearing plant to the outlet of the collection unit via the first air flow;
wherein directing, by at least a second air system, the dislodged pollen to the at least one applicator unit includes generating a second air flow, by the at least a second air system, and transporting the dislodged pollen to the at least one applicator unit via the second air flow.

15. The method of claim 14, further comprising separating, by a separating chamber, the dislodged pollen from the first air flow and directing the separated pollen to the outlet of the collection unit.

16. An assembly for collecting pollen, the assembly comprising:
a collection unit configured to dislodge pollen from pollen-bearing plants, the collection unit including a first air system configured to direct the dislodged pollen from the pollen-bearing plants to an outlet of the collection unit;
a storage unit; and
a distribution unit disposed adjacent the collection unit, the distribution unit including a second air system configured to direct the dislodged pollen from the outlet of the collection unit to the storage unit.

17. The assembly of claim 16, wherein the distribution unit includes a distributor disposed between the outlet of the collection unit and the storage unit, the distributor configured to decelerate the dislodged pollen received, via the second air system, from the outlet of the collection unit.

18. The assembly of claim 16, wherein the collection unit includes a separation chamber configured to separate the dislodged pollen from an air flow associated with the first air system and direct the separated pollen to the outlet of the collection unit.

19. The assembly of claim 18, wherein the storage unit includes the separation chamber.

20. The assembly of claim 16, further comprising at least one sensor configured to measure at least one characteristic of the pollen, in real time, as the pollen is received in the storage unit.

21. The assembly of claim 16, further comprising at least a third air system arranged in series with the second air system;
wherein the at least a third air system is configured to operate in conjunction with the second air system to direct the dislodged pollen from the outlet of the collection unit to the storage unit;
wherein the distribution unit includes a first distributor and a second distributor;
wherein the first distributor is disposed between the outlet of the collection unit and the second distributor, the first distributor configured to decelerate the dislodged pollen received, via the second air system, from the outlet of the collection unit; and
wherein the second distributor is disposed between the first distributor and the storage unit, the second distributor configured to further decelerate the dislodged pollen received, via the at least a third air system, from the first distributor of the distribution unit.
